(19) United States
(12) Patent Application Publication
(10) Pub. No.: US 2023/0374064 A1
(43) Pub. Date: Nov. 23, 2023

(54) METHODS FOR CONCENTRATING PROTEINS

(71) Applicant: Bristol-Myers Squibb Company, Princeton, NJ (US)

(72) Inventors: Jessica Joy HUNG, Marlborough, MA (US); Melissa Ann HOLSTEIN, Westford, MA (US); Shing Fung LAM, Watertown, MA (US); Sanchayita GHOSE, Acton, MA (US)

(73) Assignee: Bristol-Myers Squibb Company, Princeton, NJ (US)

(21) Appl. No.: 18/247,964

(22) PCT Filed: Oct. 4, 2021
(86) PCT No.: PCT/US2021/053394

§ 371 Date: Apr. 5, 2023

Related U.S. Application Data
(60) Provisional application No. 63/087,719, filed on Oct. 5, 2020.

(51) Int. Cl. C07K 1/34; C07K 16/28; C07K 16/22; C07K 16/24; B01D 61/14; B01D 69/02

(52) U.S. Cl. CPC C07K 1/34; C07K 16/2818; C07K 16/2827; C07K 16/2803; C07K 16/2851; C07K 16/2878; C07K 16/22; C07K 16/244; C07K 16/2866; C07K 16/2896; C07K 16/2875; B01D 61/145; B01D 69/02; C07K 2317/626; C07K 2317/35; A61K 39/00

(57) ABSTRACT

Provided herein are optimized methods for concentrating large volumes of antibody feedstocks to generate concentrated drug substances by ultrafiltration in a batch-like mode using a fed-batch setup.

### CROSS-REFERENCE TO RELATED APPLICATIONS

This application claims the priority benefit of U.S. Provisional Application No. 63/087,719, filed Oct. 5, 2020, and is hereby incorporated by reference in its entirety.

### BACKGROUND OF THE DISCLOSURE

Antibody therapies are moving increasingly towards delivery by subcutaneous formats due to the greater patient convenience and patient compliance. To enable subcutaneous delivery, the therapeutic proteins such as antibodies have to be delivered via high dosage low volume injections, which necessitates formulation of the final drug product at high concentrations. In the protein manufacturing process, the burden of generating the high concentration drug substance primarily falls on the ultrafiltration/diafiltration (UF/DF) step, where a purified protein feed stream is typically concentrated in a first ultrafiltration step to an intermediate concentration, buffer exchanged into the target formulation, and concentrated in a second ultrafiltration step to the high, final concentration. This poses unique challenges, due to the significant increase in solution viscosity with concentration as well as aggregation propensity of the proteins under prolonged exposure to shear and interfacial stresses. The high viscosities lead to high system pressures, which limit the maximum achievable protein concentration due to safety-related constraints on the system pressure. An increase in viscosity can correlate with a significant decrease in the permeate flux leading to long process times that can exacerbate the risk of protein aggregation. The generation of high concentration material results in a significant volume reduction of the load material, which can pose challenges for facility fit and capacity, as well as exacerbate aggregation challenges attributed to prolonged exposure to shear and interfacial stresses.

The need exists for improved methods to generate high concentration drug substances.

### SUMMARY OF THE DISCLOSURE

The present disclosure is related to a method of reducing a filtration process time of a protein of interest, comprising continuously loading a feed tank with a protein mixture comprising the protein of interest that has been filtered at least once (“retentate”), wherein the feed tank is separate from a main reservoir (“retentate”) tank. The present disclosure is also related to a method of concentrating a protein of interest comprising continuously loading a feed tank with a protein mixture comprising the protein of interest that has been filtered at least once (“retentate”), wherein the feed tank is separate from a main reservoir (“retentate”) tank. In some aspects, the feed tank further comprises an initial protein mixture comprising a protein of interest that has not been filtered at least once. In some aspects, the initial protein mixture and the retentate are mixed together. In some aspects, the protein mixture and/or the retentate are filtered through a filter. In some aspects, the filtered protein mixture and the retentate (“retentate”) are loaded into the feed tank. In some aspects, the loading is continued until the protein of interest is concentrated at least about 1 mg/mL, at least about 10 mg/mL, at least about 20 mg/mL, at least about 30 mg/mL, at least about 40 mg/mL, at least about 50 mg/mL, at least about 60 mg/mL, at least about 70 mg/mL, or at least about 80 mg/mL. In some aspects, the loading is continued until the protein of interest is concentrated between about 1 mg/mL and 80 mg/mL, about 5 mg/mL and 70 mg/mL, about 10 mg/mL and 60 mg/mL, about 10 mg/mL and 50 mg/mL, about 10 mg/mL and 40 mg/mL, about 10 mg/mL and 30 mg/mL, about 10 mg/mL and 20 mg/mL, about 20 mg/mL and 70 mg/mL, about 20 mg/mL and 60 mg/mL, about 20 mg/mL and 50 mg/mL, about 20 mg/mL and 40 mg/mL, or about 20 mg/mL and 30 mg/mL. In some aspects, the loading of the retentate is repeated at least twice, at least three times, at least four times, at least five times, at least six times, at least seven times, at least eight times, at least nine times, at least 10 times, at least 20 times, at least 30 times, at least 40 times, at least 50 times, at least 60 times, at least 70 times, at least 80 times, at least 90 times, at least 100 times, at least 110 times, at least 120 times, at least 130 times, at least 140 times, at least 150 times, at least 160 times, at least 170 times, at least 180 times, at least 190 times, at least 200 times, at least 210 times, at least 220 times, at least 230 times, at least 240 times, at least 250 times, at least 260 times, at least 270 times, at least 280 times, at least 290 times, or at least 300 times. In some aspects, the methods further comprise stopping the loading of the retentate to the feed tank. In some aspects, the methods further comprise directing the retentate to a reservoir tank.

The present disclosure is also related to a method of reducing a filtration process time of a protein of interest, comprising loading a protein mixture which comprises the protein of interest to a filtration system comprising a feed tank, a reservoir tank, a filter, a three way valve comprising a feed tank valve connecting the filter to the feed tank and the reservoir tank valve connecting the filter to the reservoir tank, and a reservoir input connecting the feed tank and the reservoir tank. The present disclosure is also related to a method of concentrating a protein of interest, comprising loading a protein mixture which comprises the protein of interest to a filtration system comprising a feed tank, a reservoir tank, a filter, a three way valve comprising a feed tank valve connecting the filter to the feed tank and the reservoir tank valve connecting the filter to the reservoir tank, and a reservoir input connecting the feed tank and the reservoir tank. In some aspects, the reservoir tank valve is closed until the protein of interest is sufficiently concentrated. In some aspects, the methods further comprise continually adding a protein mixture to the feed tank. In some aspects, wherein the protein mixture is directed from the feed tank to the reservoir tank. In some aspects, wherein the reservoir tank is connected to the filter. In some aspects, the filter comprises an in-line filtration membrane. In some aspects, the in-line filtration membrane is an ultrafiltration membrane. In some aspects, the in-line filtration membrane is polyvinylether, polyvinylalcohol, nylon, silicon, polysilicon, ultrananocrystalline diamond, diamond-like-carbon, silicon dioxide, titanium, silica, silicon nitride, polytetrafluorethylene, silicone, polymethacrylate, polymethyl methacrylate, polyacrylate, polystyrene, polyacrylamide, polymethacrylamide, polycarbonate, graphene, graphene oxide, polysaccharides, ceramic particles, poly(styrenedivinyl)benzene, polysulfone, polyethersulfone, modified polyethersulfone, polyarylsulfone, polyphenyl sulfphone, polyvinyl chloride, polypropylene, cellulose acetate, cellulose nitrate, polylactic acid, polyacrylonitrile, polyvinylidene fluoride, polypiperazine, polyamide-polyether block polymers, polyimide, polyetherimide, polyamide, regenerated cellulose, composite regenerated cellulose, or combinations thereof. In some aspects, the filtration membrane has a molecular weight cutoff (MWCO) lower than from about 50 kD to about 5 kD, about 50 kD, about 40 kD, about 30 kD, about 20 kD, about 10 kD, or about 5 kD. In some aspects, the MWCO is lower than about 5 kD.

In some aspects, the mixture is allowed to flow until a desired filtered protein concentration is reached. In some aspects, the desired filtered protein concentration is from about 10 mg/mL to about 300 mg/mL, e.g., about 10 mg/mL, about 50 mg/mL, about 100 mg/mL, about 110 mg/mL, about 120 mg/mL, about 130 mg/mL, about 140 mg/mL, about 150 mg/mL, about 160 mg/mL, about 170 mg/mL, about 180 mg/mL, about 190 mg/mL, about 200 mg/mL, about 250 mg/mL, or about 300 mg/mL. In some aspects, the desired filtered protein concentration is about 150 mg/mL. In some aspects, the protein viscosity is from about 0 cP to about 200 cP. In some aspects, the protein viscosity is from about 20 cP to about 60 cP. In some aspects, the the volume ratio between the volume of the feed tank and the volume of the reservoir is from about 1:2 to about 10:1, from about 1:2 to about 1:1, from about 1:1 to about 1:2, from about 1:1 about 1:3, from about 1:1 to about 1:4, from about 1:1 to about 1:5, from about 1:1 to about 1:6, from about 1:1 to about 1:7, from about 1:1 to about 1:8, from about 1:1 to about 1:9, or from about 1:1 to about 1:10. In some aspects, the volume ratio between the volume of the feed tank and the volume of the reservoir is about 1:1, about 2:1, or about 5:1. In some aspects, the protein mixture is directed to the reservoir tank and/or the filter using a diaphragm pump, rotary lobe pump, or a peristaltic pump.

In some aspects, the methods further comprise loading an initial protein mixture comprising a protein of interest that has not been filtered at least once to the feed tank prior to the continuous loading of the feed tank with the protein mixture comprising the protein of interest that has been filtered at least once (“retentate”). In some aspects, the initial protein mixture is added to the feed tank at a concentration of from about 1 mg/mL to about 30 mg/mL. In some aspects, the initial protein mixture is added to the feed tank at a concentration of about 5 mg/mL.

In some aspects, the process time is reduced by about 1%, about 5%, about 10%, about 20%, about 30%, about 40%, or about 50% as compared to a process time of a fed-batch concentration process. In some aspects, the process time is reduced by about 40% as compared to a process time of a fed-batch concentration process.

In some aspects, the process time is reduced by about 0.2 hours, about 0.4 hours, about 0.5 hours, about 0.6 hours, about 0.8 hours, or about 1.0 hours as compared to a process time of a fed-batch concentration process. In some aspects, the process time is reduced by about 0.5 hours as compared to a process time of a fed-batch concentration process.

In some aspects, the 1-2 μm particulate count is reduced by about 10%, about 20%, about 30%, about 40%, or about 50% as compared to a particulate count of a fed-batch concentration process. In some aspects, the 5-10 μm particulate count is reduced by about 10%, about 20%, about 30%, about 40%, or about 50% as compared to a particulate count of a fed-batch concentration process. In some aspects, the 10-25 μm particulate count is reduced by about 10%, about 20%, about 30%, about 40%, or about 50% as compared to a particulate count of a fed-batch concentration process.

In some aspects, the protein mixture comprises an antibody, antibody fragment, antigen-binding fragment, a fusion protein, a naturally occurring protein, a chimeric protein, or any combination thereof. In some aspects, the protein mixture comprises an antibody selected from IgM, IgA, IgE, IgD, and IgG. In some aspects, the protein mixture comprises an antibody and the antibody is an IgG antibody selected from IgG1, IgG2, IgG3, and IgG4. In some aspects, the antibody comprises a dual variable domain immunoglobulin. In some aspects, the antibody comprises a trivalent antibody. In some aspects, the antibody or antibody fragment comprises an anti-PD-1, anti-PD-L1 anti-CTLA4, anti-TIM3, anti-LAG3, anti-NKG2a, anti-ICOS, anti-CD137, anti-KIR, anti-TGFβ, anti-IL-10, anti-B7-H4, anti-GITR, anti-CXCR4, anti-CD73, anti-TIGIT, anti-OX40, anti-IL-8 antibody or antibody fragment thereof.

In some aspects, the protein mixture is derived from a bacterial, yeast, insect, or mammalian cell culture. In some aspects, the mammalian cell culture is a Chinese hamster ovary (CHO) cell culture.

In some aspects, the protein mixture is obtained from batch cell culture. In some aspects, the protein mixture is obtained from fed batch cell culture. In some aspects, the protein mixture is produced in a bioreactor. In some aspects, the protein mixture is produced in a single-use bioreactor. In some aspects, the protein mixture is obtained from perfusion cell culture. In some aspects, the protein mixture is produced in a perfusion or TFF perfusion bioreactor. In some aspects, the protein mixture is produced in a cell culture lasting from about 1 to about 60 days. In some aspects, the protein mixture is produced in a cell culture lasting about 25 days.

In some aspects, the protein mixture is added to the feed tank with a loading buffer. In some aspects, the loading buffer comprises amino acids, weak acids, weak bases, and/or sugars.

In some aspects, the methods further comprise formulating the protein into a pharmaceutical composition. In some aspects, a protein is prepared by the methods disclosed herein. In some aspects, a pharmaceutical composition comprises a protein as prepared herein.

The present disclosure is also related to a method of administering the pharmaceutical composition described herein. The present disclosure is also related to a method of treating a disease or condition in a subject in need thereof comprising administering to the subject the pharmaceutical composition.

The present disclosure is also related to a system for concentrating a protein of interest, comprising:
(a) a feed tank;
(b) a reservoir tank connected to the feed tank by a first fluid pathway;
(c) a filtration membrane connected to the reservoir tank by a second fluid pathway; and
(d) a three-way valve, wherein the three-way valve is connected to the filtration membrane by a third fluid pathway, wherein the three-way valve is connected to the reservoir tank by a fourth fluid pathway, and wherein the three-way valve is connected to the feed tank by a fifth fluid pathway,
   wherein the reservoir tank receives a protein mixture comprising the protein of interest from the feed tank via the first fluid pathway,
   wherein the filtration membrane receives the protein mixture comprising the protein of interest from the reservoir tank via the second fluid pathway and filters the protein mixture, and
   wherein the three-way valve receives retentate from the filter via the third fluid pathway and directs the retentate either to the reservoir tank via the fourth fluid pathway or to the feed tank via the fifth fluid pathway.

In some aspects, the three-way valve directs the retentate to the reservoir tank if the total volume of the protein mixture within the system is less than the capacity of the reservoir tank, and wherein the three-way valve directs the retentate to the feed tank if the total volume of the protein mixture within the system is greater than the capacity of the reservoir tank.

In some aspects, the system further comprises a sensor configured to determine the total volume and/or concentration of the protein mixture within the system, wherein the three-way valve automatically directs the retentate either to the reservoir tank or to the feed tank based on feedback from the sensor. In some aspects, the system further comprises one or more diaphram pumps, rotary lobe pumps, or peristaltic pumps. In some aspects, the filter comprises an in-line filtration membrane. In some aspects, the in-line filtration membrane is an ultrafiltration membrane. In some aspects, the in-line filtration membrane is polyvinylether, polyvinylalcohol, nylon, silicon, polysilicon, ultrananocrystalline diamond, diamond-like-carbon, silicon dioxide, titanium, silica, silicon nitride, polytetrafluorethylene, silicone, polymethacrylate, polymethyl methacrylate, polyacrylate, polystyrene, polyacrylamide, polymethacrylamide, polycarbonate, graphene, graphene oxide, polysaccharides, ceramic particles, poly(styrenedivinyl)benzene, polysulfone, polyethersulfone, modified polyethersulfone, polyarylsulfone, polyphenyl sulfphone, polyvinyl chloride, polypropylene, cellulose acetate, cellulose nitrate, polylactic acid, polyacrylonitrile, polyvinylidene fluoride, polypiperazine, polyamide-polyether block polymers, polyimide, polyetherimide, polyamide, regenerated cellulose, composite regenerated cellulose, or combinations thereof.

### BRIEF DESCRIPTION OF THE DRAWINGS

FIGS. 1A-1C show schematic diagrams of tangential flow filtration (TFF) systems. FIG. 1A shows a schematic diagram of TFF ultrafiltration/diafiltration system set up in the fed-batch configuration. FIG. 1B shows a schematic diagram of TFF ultrafiltration/diafiltration system set up in the pseudo-batch configuration. FIG. 1C shows a schematic diagram of TFF ultrafiltration/diafiltration system set up in the batch configuration.

FIGS. 2A-2B show retentate mAb concentration as a function of elapsed process time for batch loading, fed-batch loading, and pseudo-batch loading configurations. FIG. 2A shows the calculated retentate concentration of mAb A (g/L) as a function of elapsed process time (hours) time for the batch, fed-batch and pseudo-batch loading strategies. FIG. 2B shows the corresponding process times for each of stage of the process: Ultrafiltration 1 (UF1), Diafiltration (DF), and Ultrafiltration 2 (UF2), from left to right, respectively.

FIG. 3 shows permeate flux (LHM) as a function of calculated retentate concentration for mAb A during the Ultrafiltration/Diafiltration runs. The runs were performed using batch, fed-batch, or pseudo-batch loading (UF1), but all three runs were operated in the batch configuration for the DF and UF2 steps.

FIG. 4 shows the levels of high molecular weight (HMW) species for mAb A at intermediate points in the UF/DF process for the batch, fed-batch (hybrid), and pseudo-batch loading strategies.

FIGS. 5A-5D shows mAb A particle counts for particulates for the in-process UF/DF pools generated using the batch, fed-batch (hybrid), and pseudo-batch loading strategies. FIG. 5A shows the particle counts for 1-2 μm particles. FIG. 5B shows the particle counts for 5-10 μm particles. FIG. 5C shows the particle counts for 10-25 μm particles. FIG. 5D shows the particle counts for 50-100 μm particles. The particle content was quantified using microfluidic imaging. The particle counts for the 2-5 μm and 25-50 μm size ranges were omitted for brevity, but agreed with the trends observed for the 5-10 μm and 50-100 μm size ranges, respectively.

FIG. 6A shows the retentate mAb concentration as a function of process time for pseudo-batch runs using a diaphragm pump and a peristaltic pump. FIG. 6B shows the corresponding process time for each stage of the process (e.g., UF1, DF, and UF2) of FIG. 6A.

FIG. 7 shows the permeate flux as a function of retentate concentration for pseudo-batch runs using a diaphragm pump and a peristaltic pump.

FIG. 8 shows the levels of high molecular weight (HMW) species for the pseudo-batch process using a diaphragm pump and a peristaltic pump.

FIGS. 9A-9D shows mAb A particle counts for particles for the in-process UF/DF pools generated using the pseudo-batch loading strategy using either a diaphragm pump or a peristaltic pump. FIG. 9A shows the particle counts for 1-2 μm particles. FIG. 9B shows the particle counts for 5-10 μm particles. FIG. 9C shows the particle counts for 10-25 μm particles. FIG. 9D shows the particle counts for 50-100 μm particles. The particle content was quantified using microfluidic imaging.

FIG. 10A shows the retentate mAb concentration as a function of process time for pseudo-batch runs, wherein liquid volume in the retentate tank during the loading step was kept constant at a lower volume relative to the total load volume (e.g., 10% and 20%). FIG. 10B shows the corresponding process time for each stage of the process (e.g., UF1, DF, and UF2) of FIG. 10A.

FIG. 11 shows the permeate flux as a function of retentate concentration for pseudo-batch runs, wherein liquid volume in the retentate tank during the loading step was kept constant at a lower volume relative to the total load volume.

FIG. 12 shows the levels of high molecular weight (HMW) species for the pseudo-batch runs, wherein liquid volume in the retentate tank during the loading step was kept constant at a lower volume relative to the total load volume.

FIGS. 13A-13D shows mAb A particle counts for particles for the in-process UF/DF pools generated using the pseudo-batch loading, wherein liquid volume in the retentate tank during the loading step was kept constant at a lower volume relative to the total load volume. FIG. 13A shows the particle counts for 1-2 μm particles. FIG. 13B shows the particle counts for 5-10 μm particles. FIG. 13C shows the particle counts for 10-25 μm particles. FIG. 13D shows the particle counts for 50-100 μm particles. The particle content was quantified using microfluidic imaging.

FIG. 14 shows retentate protein concentration as a function of process time for a pseudo-batch and fed-batch process runs for a non-mAb therapeutic protein (MW is approximately 20 Da).

FIG. 15 shows the permeate flux as a function of retentate concentration for the pseudo-batch and fed-batch process runs shown in FIG. 14.

FIG. 16 shows the levels of high molecular weight (HMW) species for the pseudo-batch and fed-batch process runs shown in FIG. 14.

FIGS. 17A-17D shows particle counts for pseudo-batch and fed-batch process runs for a non-mAb therapeutic protein of FIG. 14. FIG. 17A shows the particle counts for 1-2 μm particles. FIG. 17B shows the particle counts for 5-10 μm particles. FIG. 17C shows the particle counts for 10-25 μm particles. FIG. 17D shows the particle counts for 50-100 μm particles. The particle content was quantified using microfluidic imaging.

### DETAILED DESCRIPTION OF THE DISCLOSURE

The present disclosure is directed to methods of reducing a filtration process time of a protein of interest. The present disclosure is also related to methods of concentrating a protein of interest.

### I. Definitions

In order that the present disclosure can be more readily understood, certain terms are first defined. As used in this specification, except as otherwise expressly provided herein, each of the following terms shall have the meaning set forth below. Additional definitions are set forth throughout the specification.

It is to be noted that the term “a” or “an” refers to one or more of that entity; for example, “a nucleotide sequence,” is understood to represent one or more nucleotide sequences. As such, the terms “a” (or “an”), “one or more,” and “at least one” can be used interchangeably herein.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range or integer range is to be understood to include the value of any integer within the recited range and, when appropriate, fractions thereof (such as one tenth and one hundredth of an integer), unless otherwise indicated.

The term “ultrafiltration” refers to, for example, a membrane-based separation process that separates molecules in solution based on size, which can accomplish separation of different molecules or accomplish concentration of like molecules.

The term “tangential flow filtration” refers to a specific filtration method in which a solute-containing solution passes tangentially across an ultrafiltration membrane and lower molecular weight solutes are passed through the membrane by applying pressure. The higher molecular weight solute-containing solution passing tangentially across the ultrafiltration membrane is retained, and thus this solution is referred to herein as “retentate.” The lower molecular weight solutes that pass through the ultrafiltration membrane are referred to herein as “permeate.” Thus, the retentate is concentrated by flowing along, e.g., tangentially, the surface of an ultrafiltration membrane under pressure. The ultrafiltration membrane has pore size with a certain cut off value. In some aspects, the cutoff value is about 50 kDa or less, e.g., 50 kDa, 40 kDa, 30 kDa, 20 kDa, or 10 Da. In some aspects, the cutoff value is 30 kD or less.

The term “diafiltration” or “DF” refers to, for example, using an ultrafiltration membrane to remove, replace, or lower the concentration of solvents, buffers, and/or salts from solutions or mixtures containing proteins, peptides, nucleic acids, or other biomolecules.

The term “fed-batch,” “fed-batch filtration” or “fed-batch filtration process” as used herein refers to a filtration (e.g., ultrafiltration) method of tangential flow filtration in which a feedstock comprising a protein of interest is loaded into a feed tank, and subsequently directed into a reservoir tank, wherein the feedstock is concentrated upon TFF and the retentate is directed back into the retentate tank. The term “batch,” “batch filtration” or “batch filtration process” refers to filtration (e.g., ultrafiltration) configuration wherein a protein mixture is loaded into a reservoir tank, from which retentate is generated and the retentate is directed back into the reservoir tank while the permeate is directed to drain for waste.

The term “polypeptide” or “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation of modification, such as conjugation with a labeling component. Also included in the definition are, for example, polypeptides containing one or more analogs of an amino acid (including, for example, unnatural amino acids, etc.), as well as other modifications known in the art. The term “polypeptide” and “protein” as used herein specifically encompass antibodies and Fc domain-containing polypeptides (e.g., immunoadhesins).

As used herein, the term “protein of interest” is used to include any protein (either natural or recombinant), present in a mixture, for which purification is desired. Such proteins of interest include, without limitation, enzymes, hormones, growth factors, cytokines, immunoglobulins (e.g., antibodies), and/or any fusion proteins. In some aspects, the protein of interest refers to any protein that can be purified and/or concentrated using the tangential flow filtration (TFF) methods described herein. In some aspects, the protein of interest is an antibody. In some aspects, the protein of interest is a recombinant protein.

The term “fed-batch culture” or “fed-batch culture process” as used herein refers to a method of culturing cells in which additional components are provided to the culture at some time subsequent to the beginning of the culture process. A fed-batch culture can be started using a basal medium. The culture medium with which additional components are provided to the culture at some time subsequent to the beginning of the culture process is a feed medium. A fed-batch culture is typically stopped at some point and the cells and/or components in the medium are harvested and optionally purified.

As used herein “perfusion” or “perfusion culture” or “perfusion culture process” refers to continuous flow of a physiological nutrient solution at a steady rate, through or over a population of cells. As perfusion systems generally involve the retention of the cells within the culture unit, perfusion cultures characteristically have relatively high cell densities, but the culture conditions are difficult to maintain and control. In addition, since the cells are grown to and then retained within the culture unit at high densities, the growth rate typically continuously decreases over time, leading to the late exponential or even stationary phase of cell growth. This continuous culture strategy generally comprises culturing mammalian cells, e.g., non-anchorage dependent cells, expressing a polypeptide and/or virus of interest during a production phase in a continuous cell culture system.

As used herein, “set point” refers to the initial setting of the condition in TFF system or other upstream processing vessel used to concentrate and/or produce protein product unless otherwise indicated. A set point is established at the outset of a UF/DF process described herein. Subsequent changes in the condition during the UF/DF after the set point can occur due to variations UF/DF conditions during TFF. For example, a set point can be a weight set point. In some aspects, a set point is a temperature set point. In some aspects, the set point can be maintained throughout the cell culturing method. In other aspects, the set point can be maintained until a different set point is set. In other aspects, the set point can be changed to another set point.

An “antibody” (Ab) shall include, without limitation, a glycoprotein immunoglobulin which binds specifically to an antigen and comprises at least two heavy (H) chains and two light (L) chains interconnected by disulfide bonds. Each H chain comprises a heavy chain variable region (abbreviated herein as V_{H}) and a heavy chain constant region. The heavy chain constant region comprises three constant domains, C_{H1}, C_{H2} and C_{H3}. Each light chain comprises a light chain variable region (abbreviated herein as V_{L}) and a light chain constant region. The light chain constant region is comprises one constant domain, C_{L}. The V_{H} and V_{L} regions can be further subdivided into regions of hypervariability, termed complementarity determining regions (CDRs), interspersed with regions that are more conserved, termed framework regions (FR). Each V_{H} and V_{L} comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. A heavy chain may have the C-terminal lysine or not. In some aspects, an antibody is a full-length antibody.

An immunoglobulin may derive from any of the commonly known isotypes, including but not limited to IgA, secretory IgA, IgG, IgD, IgE, and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes. The term “antibody” includes, by way of example, monoclonal and polyclonal antibodies; chimeric and humanized antibodies; human or nonhuman antibodies; wholly synthetic antibodies; and single chain antibodies. A nonhuman antibody may be humanized by recombinant methods to reduce its immunogenicity in man. The term “antibody” can include multivalent antibodies capable of binding more than two antigens (e.g., trivalent antibody). A trivalent antibody are IgG-shaped bispecific antibodies composed of two regular Fab arms fused via flexible linker peptides to one asymmetric third Fab-sized binding module. This third module replaces the IgG Fc region and is composed of the variable region of the heavy chain fused to CH3 with “knob”—mutations, and the variable region of the light chain fused to CH3 with matching “holes”. The hinge region does not contain disulfide bonds to facilitate antigen access to the third binding site. Where not expressly stated, and unless the context indicates otherwise, the term “antibody” includes monospecific, bispecific, or multi-specific antibodies, as well as a single chain antibody.

The term “antigen-binding portion” or “antigen-binding fragment” of an antibody, as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding fragment” of an antibody include (i) a Fab fragment (fragment from papain cleavage) or a similar monovalent fragment consisting of the VL, VH, LC and CH1 domains; (ii) a F(ab′)2 fragment (fragment from pepsin cleavage) or a similar bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; (vi) an isolated complementarity determining region (CDR) and (vii) a combination of two or more isolated CDRs which can optionally be joined by a synthetic linker. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

A “bispecific” or “bifunctional antibody” is an artificial hybrid antibody having two different heavy/light chain pairs, giving rise to two antigen binding sites with specificity for different antigens. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992).

A “fusion” or “chimeric” protein comprises a first amino acid sequence linked to a second amino acid sequence with which it is not naturally linked in nature. The amino acid sequences which normally exist in separate proteins can be brought together in the fusion polypeptide, or the amino acid sequences which normally exist in the same protein can be placed in a new arrangement in the fusion polypeptide, e.g., fusion of a Factor VIII domain of the disclosure with an Ig Fc domain. A fusion protein is created, for example, by chemical synthesis, or by creating and translating a polynucleotide in which the peptide regions are encoded in the desired relationship. A chimeric protein can further comprises a second amino acid sequence associated with the first amino acid sequence by a covalent, non-peptide bond or a non-covalent bond.

“Administering” refers to the physical introduction of a composition comprising a therapeutic agent to a subject, using any of the various methods and delivery systems known to those skilled in the art. Routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, in some embodiments, orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

“Treatment” or “therapy” of a subject refers to any type of intervention or process performed on, or the administration of an active agent to, the subject with the objective of reversing, alleviating, ameliorating, inhibiting, slowing down progression, development, severity or recurrence of a symptom, complication or condition, or biochemical indicia associated with a disease. Response Evaluation Criteria In Solid Tumors (RECIST) is a measure for treatment efficacy and are established rules that define when tumors respond, stabilize, or progress during treatment. RECIST 1.1 is the current guideline to solid tumor measurement and definitions for objective assessment of change in tumor size for use in adult and pediatric cancer clinical trials. Eastern Cooperative Oncology Group (ECOG) Performance Status is a numbering scale used to define the population of patients to be studied in a trial, so that it can be uniformly reproduced among physicians who enroll patients. In pediatric patients, the Lansky Performance Scale is a method for describing functional status in children. It was derived and internally validated in children with cancer to assess response to therapies and overall status.

As described herein, any concentration range, percentage range, ratio range, or integer range is to be understood to include the value of any integer within the recited range and, when appropriate, fractions thereof (such as one tenth and one hundredth of an integer), unless otherwise indicated.

The term “desired final protein concentration” as used herein refers to the protein concentration of a protein of interest that has been concentrated using the tangential flow filtration methods described herein. For example, a desired final protein concentration for a protein of interest is achieved by subjecting a mixture comprising the protein of interest to the UF1, DF, and UF2 steps as described herein. In some aspects, the desired final protein concentration is up to 300 mg/mL.

As used herein, “pharmaceutically acceptable carrier” refers to a vehicle for a pharmacologically active agent. The carrier facilitates delivery of the active agent to the target site without terminating the function of the agent. Non-limiting examples of suitable forms of the carrier include solutions, creams, gels, gel emulsions, jellies, pastes, lotions, salves, sprays, ointments, powders, solid admixtures, aerosols, emulsions (e.g., water in oil or oil in water), gel aqueous solutions, aqueous solutions, suspensions, liniments, tinctures, and patches suitable for topical administration.

As used herein, the phrase “pharmaceutically acceptable composition” (or “pharmaceutical composition”) refers to a composition that is acceptable for pharmaceutical administration, such as to a human being. Such a composition can include substances with impurities at a level not exceeding an acceptable level for pharmaceutical administration (such level including an absence of such impurities), and can include pharmaceutically acceptable excipients, vehicles, carriers and other inactive ingredients, for example, to formulate such composition for ease of administration, in addition to any active agent(s). For example, a pharmaceutically acceptable anti-PD1 antibody composition can include DNA, so long as it is at a level acceptable for administration to humans.

### II. Ultrafiltration Methods

The present disclosure provides a protein purification method that enables the final protein yield to be highly concentrated. In some aspects, the methods of the present disclosure reduce the process time required for a first ultrafiltration step (e.g., in a process comprising or consisting of a first ultrafiltration step, diafiltration, and a second ultrafiltration step) for protein purification processes where the feedstock undergoes a large volume reduction, and the initial volume is large enough to require operating initially in fed-batch mode.

In some aspects, the methods of the present disclosure mitigate indirect challenges associated with generation of high concentration drug substances (e.g., protein of interest) by ultrafiltration. For example, the generation of high concentration material (e.g., approximately 150-300 g/L) results in a significant volume reduction of the load material, which poses challenges for facility fit and capacity, in addition to exacerbating aggregation challenges attributed to prolonged exposure to shear and interfacial stresses. During ultrafiltration, sample volume decreases significantly (e.g., typically 10-fold or more) while the retentate vessel and system holdup volumes are fixed, resulting in a point in the process where there is a large mismatch between the scales of the system (e.g., retentate vessel and system holdup volume) and a sample volume. Because the system flow path, including the bottom part of the vessel, must remain filled with liquid throughout the filtration process, the drug substance (e.g., protein of interest) volume at the final concentration combined with the retentate vessel geometry dictates a minimum working volume for the system. Because the minimum working volume increases with vessel size, the final drug substance sets an upper limit of the retentate vessel size. This upper limit is lower than the load volume. As such, part of the load material needs to be fed into the retentate vessel using fed-batch loading. The percentage of the initial load volume that must be fed in by fed-batch loading is a function of facility fit, such as the available vessel sizes and system holdup volume.

While fed-batch loading allows for processing of large load volumes, this loading strategy imposes a process time penalty compared to batch operation. For a given amount of remaining sample volume, the retentate protein concentration is higher for a fed-batch process than for a batch process, since the liquid (buffer) volume of the sample is split between the dilute material in the load vessel (e.g., feed tank) and the concentrated protein in the retentate vessel in fed-batch, rather than distributed homogeneously among the total protein mass (e.g., everything in the retentate vessel) in batch. As a result, the fed-batch permeate flux is lower due to the higher retentate concentration. The system therefore operates at a higher concentration and lower flux for a large part of the first ultrafiltration step in fed-batch mode compared to batch mode, leading to a longer process time, which in turn increases the risk of shear and interfacial stress-induced aggregation.

In manufacturing-scale setups, the operation mode of the first ultrafiltration step for generating high concentration drug substances is typically neither fully batch nor fed-batch. Instead, the ultrafiltration step operates in fed-batch mode during feeding of the load material into the retentate vessel, and then switches to batch mode once the load material is fully contained in the retentate vessel. This mixed-mode ultrafiltration step will hereafter be referred to as the “hybrid” mode. The hybrid mode results in a process time for the first ultrafiltration step that falls between the process times expected for operating fully in batch mode or fed-batch mode, and which depends on the crossover point where the system switches from operating in fed-batch mode to batch mode. The equations describing the crossover point and hybrid process times are described in Example 1. The concentration corresponding to the crossover point in turn is a function of the relative load and system volumes, making the process time a strong function of facility fit.

This dependency creates challenges for process development and technology transfer, as the process parameter specifications developed at laboratory scale can lead to unexpectedly longer process times at the manufacturing scale if the differences in the relative load and system volumes are not considered during technology transfer. In contrast, the process time of a batch process is only dependent on the membrane loading, making the process fully scalable.

No systemic approach has been developed to address the process time penalty or facility fit dependency resulting from the hybrid operation mode during the first ultrafiltration step. To mitigate these challenges, the methods disclosed herein were developed to allow for batch-like operation of the ultrafiltration step using a fed-batch-like setup, referred to herein as the pseudo-batch operation mode.

The present disclosure provides optimized methods for concentrating large volumes of protein feedstock to generate concentrated drug substances by ultrafiltration in a batch-like mode using a fed-batch setup. The present disclosure also relates to methods for generating solutions comprising highly concentrated proteins by tangential flow filtration (TFF). The methods disclosed herein reduce the process time required for a first ultrafiltration step (e.g., in a process comprising or consisting of a first ultrafiltration step, diafiltration, and a second ultrafiltration step) for processes where the feedstock undergoes a large volume reduction, and the initial volume is large enough to require operating initially in a fed-batch mode. In some aspects, the methods improve product quality, as quantified by particulate and impurity burden generated during the ultrafiltration process, by virtue of shorter process time. In some aspects, the methods eliminate the variability in process time observed between equipment setups between laboratory, e.g., development, and manufacturing scales when operating in fed-batch mode. The resulting improved consistency in process times between scales, in some aspects, can improve the accuracy of scale-down ultrafiltration/diafiltration (UF/DF) models, leading to more efficient scale-up and technology transfer campaigns.

The present disclosure is directed to a pseudo-batch configuration for UF/DF that, in some aspects, can mitigate the time penalty associated with fed-batch loading by converting the fed-batch setup (e.g., using a feed tank and retentate vessel) into a batch-like operation (e.g., connecting the feed tank and retentate vessel as described herein such that they function as a single vessel in a Tangential Flow Filtration (TFF) recirculation loop) to concentrate a protein of interest.

Tangential flow filtration is an ultrafiltration procedure that relies on the use of fluid pressure to drive the migration of the smaller molecules through an ultrafiltration membrane while simultaneously retaining larger molecules (e.g., the “retentate”). In general, a membrane with a molecular weight cut-off (MWCO) is selected that is three to six times smaller than the molecular weight of the protein to be retained. Other factors known to a person in the art can also impact the selection of the appropriate MWCO, e.g. flow rate, processing time, transmembrane pressure, molecular shape or structure, solute concentration, presence of other solutes, and ionic conditions.

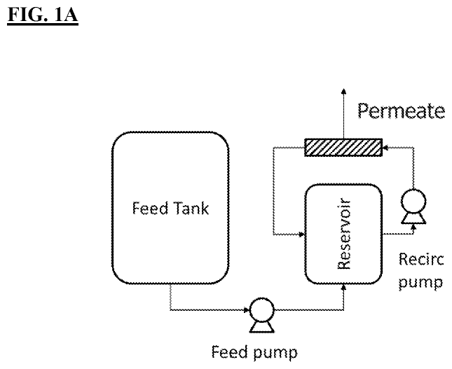

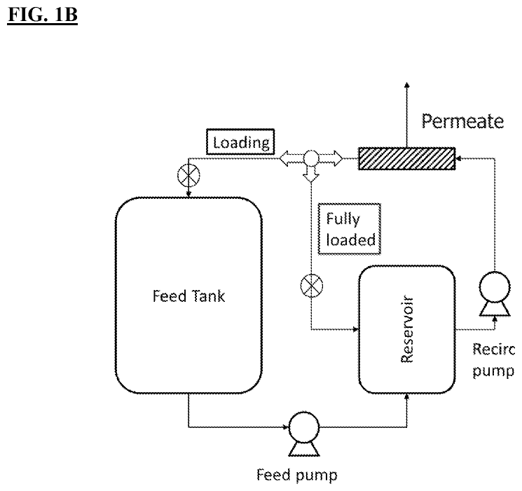

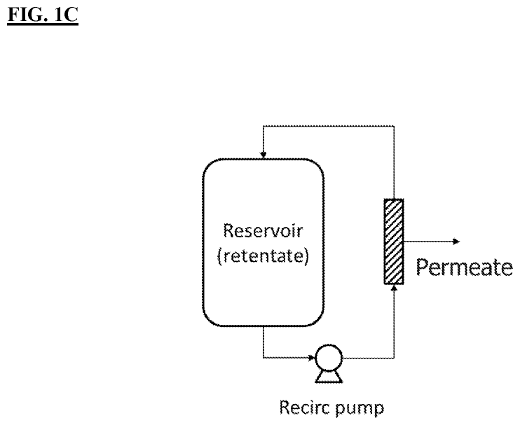

The traditional fed-batch TFF configuration is shown in FIG. 1A, wherein the feed tank holding the load material is connected to the retentate vessel (e.g., reservoir) by a feed pump, and the retentate vessel is separately incorporated into a recirculation loop with a TFF membrane device and recirculation pump.

In some aspects, the methods of concentrating and/or reducing the filtration time for a protein of interest by TFF include a first ultrafiltration step (UF1), diafiltration (DF), and a second ultrafiltration step (UF2).

In the pseudo-batch configuration described herein, in some aspects, the flow path is modified to incorporate the feed tank into the recirculation loop (FIG. 1B). A three-way valve is placed after the retentate port of the TFF filter module to direct the retentate flow to either the feed tank or retentate vessel depending on the status of the first ultrafiltration step. In some aspects, mixers are used for both the feed tank and the reservoir (e.g., retentate) tank.

In some aspects, during the initial loading stage of UF1, the three-way valve is set to direct the retentate flow to the feed tank and block the flow to the retentate vessel. The load material is fed from the feed tank into the retentate vessel with the feed pump, but the retentate from the TFF filter module is returned to the feed tank and mixed with the remaining load material. This new load material mixture is now slightly more concentrated, and fed to the retentate vessel, and the cycle repeats. In some aspects, the protein solution in both the feed tank and retentate vessel are concentrated at the same rate, unlike in fed-batch loading where the retentate becomes increasingly more concentrated while the load material remains fixed at the initial dilute concentration. With this configuration, the feed tank effectively acts as an extension of the retentate vessel, and the two act as a single reservoir in a recirculation loop, similar to a batch set up (FIG. 1C). The pseudo-batch configuration described herein converts the otherwise hybrid-mode process to a batch-like process, mitigating the time penalty for the fed-batch loading portion of the first ultrafiltration step as well as the facility fit dependency of the process time. In some aspects, during the loading step, the liquid volume in the retentate tank is kept constant at a lower volume relative to the total load volume. In some aspects, the liquid volume is kept constant at about 10%, about 15%, about 20%, about 25%, about 30%, about 35%, or about 40% of the total initial load volume during the loading step. In some aspects, the liquid volume is kept constant at between about 5% to about 10%, about 5% to about 15%, about 5% to about 20%, about 5% to about 25%, about 5% to about 30%, about 10% to about 15%, about 10% to about 20%, about 10% to about 25%, about 10% to about 30%, about 15% to about 20%, about 15% to about 25%, about 15% to about 30%, about 20% to about 30%, or about 25% to about 30% of the total initial load volume during the loading step. In some aspects, the liquid volume is kept constant at about 10% of the total initial load volume during the loading step. In some aspects, the liquid volume is kept constant at about 20% of the total initial load volume during the loading step.

In some aspects, when the loading step is complete (e.g., when the total protein solution volume has decreased to below the retentate vessel volume), the three-way valve is actuated to re-direct the retentate flow to the retentate vessel and block flow to the feed tank, effectively eliminating the feed tank from the recirculation loop and converting the TFF setup to a true batch configuration. In some aspects, the feed pump can be operated for additional time to perform an air chase (e.g., into the retentate vessel) of the residual material in the connecting tubing between the two vessels in order to maximize recovery of the load material.

A TFF membrane for concentration can be selected based on its rejection characteristics for the sample to be concentrated. As a general rule, the molecular weight cut-off (MWCO) of the membrane should be ⅓^{rd} to ⅙^{th} the molecular weight of the molecule to be retained (e.g., the protein of interest), to assure complete retention. The closer the MWCO is to that of the sample, the greater the risk for some product loss during concentration. The risk increases if diafiltration will also be used since the relative loss depends on the total volume of filtrate that will be generated. Membrane flux rate (filtrate flow rate per unit area of membrane) is related to pore size. The smaller the pores, the lower the flux rate for the same applied pressure. Therefore, when selecting a membrane for concentration/diafiltration, one must consider the time factor versus product recovery. The process time can be reduced by increasing the amount of membrane area used.

Diafiltration is a technique that uses a filtration membrane (e.g., ultrafiltration membrane) to completely remove, replace, or lower the concentration of salts or solvents from solutions containing proteins, peptides nucleic acids, and other biomolecules. DF selectively utilizes permeable (e.g., porous) membrane filters to separate the components of solutions and suspensions based on their molecular size. An ultrafiltration membrane retains molecules that are larger than the pores of the membrane while smaller molecules such as salts, solvents and water, which are 100% permeable, freely pass through the membrane. DF is a fractionation process that washes smaller molecules through a membrane and retains larger molecules (e.g., the protein of interest) in the retentate without ultimately changing concentration.

Diafiltration can be continuous or discontinuous. In continuous diafiltration, the diafiltration solution (e.g., buffer) is added to the sample feed reservoir at the same rate as filtrate is generated. In this way, the volume in the sample reservoir remains constant, but the small molecules (e.g., salts) that can freely permeate through the membrane are washed away. Using salt removal as an example, each additional diafiltration volume (DV) reduces the salt concentration further. (DV is a measure of the extent of washing that has been performed during a DF step. It is based on the volume of diafiltration buffer introduced compared to the retentate volume. In a constant-volume DF, the retentate volume is held constant and a DF buffer enters at the same rate that permeate leaves. For example, one diafiltration volume is equal to adding a volume of buffer to the feed reservoir equal to the volume of product in the system, then concentrating back to the starting volume. For example, a 200 mL sample to start, one diafiltration volume, (DV 1) is equal to 200 mL). A second diafiltration volume (DV 2) will reduce the ionic strength by ˜99% with continuous diafiltration. In discontinuous diafiltration, the solution is first diluted and then concentrated back to the starting volume. The process is then repeated until the required concentration of small molecules (e.g., salts) remaining in the reservoir is reached. Each additional DV reduces the salt concentration further. Continuous diafiltration requires less filtrate volume to achieve the same degree of salt reduction as discontinuous diafiltration. By first concentrating a sample, the amount of diafiltration solution required to achieve a specified ionic strength can be substantially reduced.

In some aspects a DF feed pump will run only during a DF or recover mode if the retentate vessel weight is below a retentate vessel weight setpoint. In some aspects, this weight is checked by a control system every 2 seconds. In some aspects, the DF weight is set and the DF pump will maintain until DF end point. In some aspects, the DF pump will turn on if the vessel weight drops below an entered set point. After the DF endpoint is reached, the system will progress to the concentration step. In some aspects, the DF endpoint choice is selected through a graphical user interface of the control system. In some aspects, the default end point choice is air in the line. In some aspects, the DF weight set point would be the full retentate vessel weight.

The present disclosure provide methods of reducing a filtration process time of a protein of interest, comprising continuously loading a feed tank with a protein mixture comprising the protein of interest that has been filtered at least once (e.g., retentate), wherein the feed tank is separate from a main reservoir (e.g., retentate) tank. In some aspects, the method of reducing a filtration process time of a protein of interest, comprises loading a protein mixture which comprises the protein of interest to a filtration system comprising a feed tank, a reservoir tank, a filter, a three way valve comprising a feed tank valve connecting the filter to the feed tank and the reservoir tank valve connecting the filter to the reservoir tank, and a reservoir input connecting the feed tank and the reservoir tank.

The present disclosure also provides methods of concentrating a protein of interest comprising continuously loading a feed tank with a protein mixture comprising the protein of interest that has been filtered at least once (e.g., retentate), wherein the feed tank is separate from a main reservoir (e.g., retentate) tank. In some aspects, the method of concentrating a protein of interest comprises loading a protein mixture which comprises the protein of interest to a filtration system comprising a feed tank, a reservoir tank, a filter, a three way valve comprising a feed tank valve connecting the filter to the feed tank and the reservoir tank valve connecting the filter to the reservoir tank, and a reservoir input connecting the feed tank and the reservoir tank.

In some aspects, the feed tank further comprises an initial protein mixture comprising a protein of interest that has not been filtered at least once, wherein the initial protein mixture and the retentate are mixed together. In some aspects, the protein mixture and the retentate are filtered through a filter (e.g., ultrafiltration filter). In some aspects, the filtered protein mixture and the retentate are loaded into the feed tank. In some aspects, the protein mixture and retentate are loaded into the feed tank continuously until the protein of interest is concentrated at least about 1 mg/mL, at least about 10 mg/mL, at least about 20 mg/mL, at least about 30 mg/mL, at least about 40 mg/mL, at least about 50 mg/mL, at least about 60 mg/mL, at least about 70 mg/mL, or at least about 80 mg/mL. In some aspects, the protein mixture and retentate are loaded into the feed tank continuously until the protein of interest is concentrated at least about 1 mg/mL, at least about 5 mg/mL, at least about 10 mg/mL, at least about 11 mg/mL, at least about 12 mg/mL, at least about 13 mg/mL, at least about 14 mg/mL, at least about 15 mg/mL, at least about 16 mg/mL, at least about 17 mg/mL, at least about 18 mg/mL, at least about 19 mg/mL, at least about 20 mg/mL. In some aspects, the protein mixture and retentate are loaded into the feed tank continuously until the protein of interest is concentrated at least about 21 mg/mL, at least about 22 mg/mL, at least about 23 mg/mL, at least about 24 mg/mL, at least about 25 mg/mL, at least about 26 mg/mL, at least about 27 mg/mL, at least about 28 mg/mL, at least about 29 mg/mL, or at least about 30 mg/mL. In some aspects, the protein mixture and retentate are loaded into the feed tank continuously until the protein of interest is concentrated at least about 31 mg/mL, at least about 32 mg/mL, at least about 33 mg/mL, at least about 34 mg/mL, at least about 35 mg/mL, at least about 36 mg/mL, at least about 37 mg/mL, at least about 38 mg/mL, at least about 39 mg/mL, at least about 40 mg/mL, at least about 45 mg/mL, at least about 50 mg/mL, at least about 55 mg/mL, at least about 60 mg/mL, at least about 65 mg/mL, at least about 70 mg/mL, at least about 75 mg/mL, or at least about 80 mg/mL, at least about 85 mg/mL, or at least about 90 mg/mL. In some aspects, the protein mixture and retentate are loaded into the feed tank continuously until the protein of interest is concentrated between about 1 mg/mL and 80 mg/mL, about 5 mg/mL and 70 mg/mL, about 10 mg/mL and 60 mg/mL, about 10 mg/mL and 50 mg/mL, about 10 mg/mL and 40 mg/mL, about 10 mg/mL and 30 mg/mL, about 10 mg/mL and 20 mg/mL, about 20 mg/mL and 70 mg/mL, about 20 mg/mL and 60 mg/mL, about 20 mg/mL and 50 mg/mL, about 20 mg/mL and 40 mg/mL, or about 20 mg/mL and 30 mg/mL. In some aspects, the protein mixture and retentate are loaded into the feed tank continuously until the protein of interest is concentrated between about 1 mg/mL and 10 mg/mL, about 10 mg/mL and 20 mg/mL, about 20 mg/mL and 30 mg/mL, about 30 mg/mL and 40 mg/mL, about 40 mg/mL and 50 mg/mL, about 50 mg/mL and 60 mg/mL, about 60 mg/mL and 70 mg/mL, or about 70 mg/mL and 80 mg/mL.

In some aspects, the loading of the retentate is recirculated through the pseudo-batch flow path described herein. In some aspects, the loading of the retentate is repeated at least twice, at least three times, at least four times, at least five times, at least six times, at least seven times, at least eight times, at least nine times, at least 10 times, at least 20 times, at least 30 times, at least 40 times, at least 50 times, at least 60 times, at least 70 times, at least 80 times, at least 90 times, at least 100 times, at least 110 times, at least 120 times, at least 130 times, at least 140 times, at least 150 times, at least 160 times, at least 170 times, at least 180 times, at least 190 times, at least 200 times, at least 210 times, at least 220 times, at least 230 times, at least 240 times, at least 250 times, at least 260 times, at least 270 times, at least 280 times, at least 290 times, or at least 300 times.

In some aspects, the method further comprises stopping the loading of the retentate to the feed tank. In some aspects, the method further comprises directing the retentate to a reservoir tank. In some aspects, the reservoir tank valve is closed until the protein of interest is sufficiently concentrated. In some aspects, the method further comprises continually adding a protein mixture to the feed tank. In some aspects, the protein mixture is directed from the feed tank to the reservoir tank.

In some aspects, the reservoir tank is connected to the filter. In some aspects, the filter comprises an in-line filtration membrane. In some aspects, the in-line filtration membrane is an ultrafiltration membrane. In some aspects, the in-line filtration membrane is polyvinylether, polyvinylalcohol, nylon, silicon, polysilicon, ultrananocrystalline diamond, diamond-like-carbon, silicon dioxide, titanium, silica, silicon nitride, polytetrafluorethylene, silicone, polymethacrylate, polymethyl methacrylate, polyacrylate, polystyrene, polyacrylamide, polymethacrylamide, polycarbonate, graphene, graphene oxide, polysaccharides, ceramic particles, poly(styrenedivinyl)benzene, polysulfone, polyethersulfone, modified polyethersulfone, polyarylsulfone, polyphenyl sulfphone, polyvinyl chloride, polypropylene, cellulose acetate, cellulose nitrate, polylactic acid, polyacrylonitrile, polyvinylidene fluoride, polypiperazine, polyamide-polyether block polymers, polyimide, polyetherimide, polyamide, regenerated cellulose, composite regenerated cellulose, or combinations thereof. In some aspects, the in-line filtration membrane is polyethersulfone. In some aspects, the in-line filtration membrane is cellulose. In some aspects, the in-line filtration membrane is a combination of polyethersulfone and cellulose. In some aspects, filtration membrane has a molecular weight cutoff (MWCO) lower than from about 50 kD to about 5 kD. In some aspects, the filtration membrane has a MWCO lower than about 5 kD.

In some aspects, the mixture is allowed to flow (e.g., recirculate) until a desired filtered protein concentration is reached. In some aspects, the desired filtered protein concentration is from about 10 mg/mL to about 300 mg/mL. In some aspects, the desired filtered protein concentration is from about 20 mg/mL to about 300 mg/mL. In some aspects, the desired filtered protein concentration is from about 30 mg/mL to about 300 mg/mL. In some aspects, the desired filtered protein concentration is from about 40 mg/mL to about 300 mg/mL. In some aspects, the desired filtered protein concentration is from about 50 mg/mL to about 300 mg/mL. In some aspects, the desired filtered protein concentration is from about 60 mg/mL to about 300 mg/mL. In some aspects, the desired filtered protein concentration is from about 70 mg/mL to about 300 mg/mL. In some aspects, the desired filtered protein concentration is from about 80 mg/mL to about 300 mg/mL. In some aspects, the desired filtered protein concentration is from about 90 mg/mL to about 300 mg/mL. In some aspects, the desired filtered protein concentration is from about 100 mg/mL to about 300 mg/mL. In some aspects, the desired filtered protein concentration is from about 110 mg/mL to about 300 mg/mL. In some aspects, the desired filtered protein concentration is from about 120 mg/mL to about 300 mg/mL. In some aspects, the desired filtered protein concentration is from about 130 mg/mL to about 300 mg/mL. In some aspects, the desired filtered protein concentration is from about 140 mg/mL to about 300 mg/mL. In some aspects, the desired filtered protein concentration is from about 150 mg/mL to about 300 mg/mL. In some aspects, the desired filtered protein concentration is from about 160 mg/ml to about 300 mg/mL. In some aspects, the desired filtered protein concentration is from about 170 mg/mL to about 300 mg/mL. In some aspects, the desired filtered protein concentration is from about 180 mg/mL to about 300 mg/mL. In some aspects, the desired filtered protein concentration is from about 190 mg/mL to about 300 mg/mL. In some aspects, the desired filtered protein concentration is from about 200 mg/mL to about 300 mg/mL. In some aspects, the desired filtered protein concentration is from about 210 mg/mL to about 300 mg/mL. In some aspects, the desired filtered protein concentration is from about 220 mg/mL to about 300 mg/mL. In some aspects, the desired filtered protein concentration is from about 230 mg/mL to about 300 mg/mL. In some aspects, the desired filtered protein concentration is from about 240 mg/mL to about 300 mg/mL. In some aspects, the desired filtered protein concentration is from about 250 mg/mL to about 300 mg/mL. In some aspects, the desired filtered protein concentration is from about 260 mg/mL to about 300 mg/ml. In some aspects, the desired filtered protein concentration is from about 270 mg/mL to about 300 mg/mL. In some aspects, the desired filtered protein concentration is from about 280 mg/ml to about 300 mg/mL. In some aspects, the desired filtered protein concentration is from about 290 mg/mL to about 300 mg/mL. In some aspects, the desired filtered protein concentration is about 150 mg/mL.

In some aspects, the protein viscosity is from about 0 cP to about 200 cP. In some aspects, the protein viscosity is from about 20 cP to about 60 cP. In some aspects, the protein viscosity is from about 10 cP to about 200 cP. In some aspects, the protein viscosity is from about 20 cP to about 200 cP. In some aspects, the protein viscosity is from about 30 cP to about 200 cP. In some aspects, the protein viscosity is from about 40 cP to about 200 cP. In some aspects, the protein viscosity is from about 50 cP to about 200 cP. In some aspects, the protein viscosity is from about 60 cP to about 200 cP. In some aspects, the protein viscosity is from about 70 cP to about 200 cP. In some aspects, the protein viscosity is from about 80 cP to about 200 cP. In some aspects, the protein viscosity is from about 90 cP to about 200 cP. In some aspects, the protein viscosity is from about 100 cP to about 200 cP. In some aspects, the protein viscosity is from about 100 cP to about 200 cP. In some aspects, the protein viscosity is from about 120 cP to about 200 cP. In some aspects, the protein viscosity is from about 130 cP to about 200 cP. In some aspects, the protein viscosity is from about 140 cP to about 200 cP. In some aspects, the protein viscosity is from about 150 cP to about 200 cP. In some aspects, the protein viscosity is from about 160 cP to about 200 cP. In some aspects, the protein viscosity is from about 170 cP to about 200 cP. In some aspects, the protein viscosity is from about 180 cP to about 200 cP. In some aspects, the protein viscosity is from about 190 cP to about 200 cP.

In some aspects, the volume ratio between the volume of the feed tank and the volume of the reservoir is from about 1:2 to about 10:1, from about 1:2 to about 1:1, from about 1:1 to about 1:2, from about 1:1 about 1:3, from about 1:1 to about 1:4, from about 1:1 to about 1:5, from about 1:1 to about 1:6, from about 1:1 to about 1:7, from about 1:1 to about 1:8, from about 1:1 to about 1:9, or from about 1:1 to about 1:10. In some aspects, the volume ratio between the volume of the feed tank and the volume of the reservoir tank is about 1:1, about 2:1, or about 5:1.

In some aspects, the protein mixture is directed to the reservoir tank and/or the filter using a diaphragm pump, rotary lobe pump, or a peristaltic pump. In some aspects, the protein mixture is directed to the reservoir tank and/or the filter using a diaphragm pump. In some aspects, the protein mixture is directed to the reservoir tank and/or the filter using a peristaltic pump.

In some aspects, the methods of concentrating a protein of interest and/or reducing a filtration process time for a protein of interest further comprise loading an initial protein mixture comprising a protein of interest that has not been filtered at least once to the feed tank prior to the continuous loading of the feed tank with the protein mixture comprising the protein of interest that has been filtered at least once (“retentate”). In some aspects, the initial protein mixture is added to the feed tank at a concentration of from about 1 mg/mL to about 30 mg/mL. In some aspects, the initial protein mixture is added to the feed tank at a concentration of about 5 mg/mL.

In some aspects, the process time is reduced by about 1%, about 5%, about 10%, about 20%, about 30%, about 40%, or about 50% as compared to a process time of a fed-batch concentration process. In some aspects, the process time is reduced by about 40% as compared to a process time of a fed-batch concentration process. In some aspects, the process time is reduced by about 0.2 hours, about 0.4 hours, about 0.5 hours, about 0.6 hours, about 0.8 hours, or about 1.0 hours as compared to a process time of a fed-batch concentration process. In some aspects, the process time is reduced by about 0.5 hours as compared to a process time of a fed-batch concentration process.

In some aspects, wherein the 1-2 μparticulate count is reduced by about 10%, about 20%, about 30%, about 40%, or about 50% as compared to a particulate count of a fed-batch concentration process. In some aspects, the 5-10 μparticulate count is reduced by about 10%, about 20%, about 30%, about 40%, or about 50% as compared to a particulate count of a fed-batch concentration process. In some aspects, the 10-25 μparticulate count is reduced by about 10%, about 20%, about 30%, about 40%, or about 50% as compared to a particulate count of a fed-batch concentration process.

In some aspects, the protein mixture is added to the feed tank with a loading buffer. In some aspects, the loading buffer comprises amino acids, weak acids, weak bases, and/or sugars.

### III. Protein of Interest

In some aspects, the methods disclosed herein can be applied to any protein product (e.g., a protein of interest). In some aspects, the protein product is a therapeutic protein. In some aspects, the therapeutic protein is selected from an antibody or antigen-binding fragment thereof, an Fc fusion protein, an anticoagulant, a blood clotting factor, a bone morphogenic protein, an engineered protein scaffold, an enzyme, a growth factor, a hormone, an interferon, an interleukin, and a thrombolytic. In some aspects, the protein product is an antibody or antigen-binding fragment thereof. In some aspects, the protein is a recombinant protein.

In some aspects, the protein product is an antibody or an antigen binding fragment thereof. In some aspects, the protein product is a chimeric polypeptide comprising an antigen binding fragment of an antibody. In some aspects, the protein product is a monoclonal antibody or an antigen binding fragment thereof (“mAb”). The antibody can be a human antibody, a humanized antibody, or a chimeric antibody. In some aspects, the protein product is a bispecific antibody.

In some aspects, the mixture comprising the protein product and the contaminant comprises a product of a prior purification step. In some aspects, the mixture is the raw product of a prior purification step. In some aspects, the mixture is a solution comprising the raw product of a prior purification step and a buffer, e.g., the starting buffer. In some aspects, the mixture comprises the raw product of a prior purification step reconstituted in the starting buffer.

In some aspects, the source of the protein product is bulk protein. In some aspects, the source of the protein product is a composition comprising protein product and non-protein components. The non-protein components can include DNA and other contaminants.

In some aspects, the source of the protein product is from an animal. In some aspects, the animal is a mammal such as a non-primate (e.g., cow, pig, horse, cat, dog, rat etc.) or a primate (e.g., monkey or human). In some aspects, the source is tissue or cells from a human. In certain aspects, such terms refer to a non-human animal (e.g., a non-human animal such as a pig, horse, cow, cat or dog). In some aspects, such terms refer to a pet or farm animal. In some aspects, such terms refer to a human.

In some aspects, the protein products purified by the methods described herein are fusion proteins. A “fusion” or “fusion protein” comprises a first amino acid sequence linked in frame to a second amino acid sequence with which it is not naturally linked in nature. The amino acid sequences which normally exist in separate proteins can be brought together in a fusion polypeptide, or the amino acid sequences which normally exist in the same protein can be placed in a new arrangement in the fusion polypeptide. A fusion protein is created, for example, by chemical synthesis, or by creating and translating a polynucleotide in which the peptide regions are encoded in the desired relationship. A fusion protein can further comprise a second amino acid sequence associated with the first amino acid sequence by a covalent, non-peptide bond or a non-covalent bond. Upon transcription/translation, a single protein is made. In this way, multiple proteins, or fragments thereof can be incorporated into a single polypeptide. “Operably linked” is intended to mean a functional linkage between two or more elements. For example, an operable linkage between two polypeptides fuses both polypeptides together in frame to produce a single polypeptide fusion protein. In a particular aspect, the fusion protein further comprises a third polypeptide which, as discussed in further detail below, can comprise a linker sequence.

In some aspects, the proteins purified by the methods described herein are antibodies. Antibodies can include, for example, monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, single domain antibodies, monovalent antibodies, single chain antibodies or single-chain Fvs (scFv), camelized antibodies, affibodies, Fab fragments, F(ab′)2 fragments, disulfide-linked Fvs (sdFv), anti-idiotypic (anti-Id) antibodies (including, e.g., anti-anti-Id antibodies), and antigen-binding fragments of any of the above. In some aspects, antibodies described herein refer to polyclonal antibody populations. Antibodies can be of any type (e.g., IgG, IgE, IgM, IgD, IgA or IgY), any class (e.g., IgG1, IgG2, IgG3, IgG4, IgA1 or IgA2), or any subclass (e.g., IgG2a or IgG2b) of immunoglobulin molecule. In some aspects, antibodies described herein are IgG antibodies, or a class (e.g., human IgG1 or IgG4) or subclass thereof. In a aspects, the antibody is a humanized monoclonal antibody. In some aspects, the antibody is a human monoclonal antibody, preferably that is an immunoglobulin. In some aspects, an antibody described herein is an IgG1, or IgG4 antibody.

In some aspects, the protein is an anti-LAG3 antibody, an anti-CTLA-4 antibody, an anti-TIM3 antibody, an anti-NKG2a antibody, an anti-ICOS antibody, an anti-CD137 antibody, an anti-KIR antibody, an anti-TGFβ antibody, an anti-IL-10 antibody, an anti-B7-H4 antibody, an anti-Fas ligand antibody, an anti-mesothelin antibody, an anti-CD27 antibody, an anti-GITR antibody, an anti-CXCR4 antibody, an anti-CD73 antibody, an anti-TIGIT antibody, an anti-OX40 antibody, an anti-PD-1 antibody, an anti-PD-L1 antibody, an anti-IL8 antibody, or any combination thereof. In some aspects, the protein is Abatacept NGP. In other aspects, the protein is Belatacept NGP.

In some aspects, the protein is an anti-PD-1 antibody.

Anti-PD-1 antibodies that are known in the art can be used in the presently described compositions and methods. Various human monoclonal antibodies that bind specifically to PD-1 with high affinity have been disclosed in U.S. Pat. No. 8,008,449. Anti-PD-1 human antibodies disclosed in U.S. Pat. No. 8,008,449 have been demonstrated to exhibit one or more of the following characteristics: (a) bind to human PD-1 with a K_{D} of 1×10⁻⁷ M or less, as determined by surface plasmon resonance using a Biacore biosensor system; (b) do not substantially bind to human CD28, CTLA-4 or ICOS; (c) increase T-cell proliferation in a Mixed Lymphocyte Reaction (MLR) assay; (d) increase interferon-γ production in an MLR assay; (e) increase IL-2 secretion in an MLR assay; (f) bind to human PD-1 and cynomolgus monkey PD-1; (g) inhibit the binding of PD-L1 and/or PD-L2 to PD-1; (h) stimulate antigen-specific memory responses; (i) stimulate antibody responses; and (j) inhibit tumor cell growth in vivo. Anti-PD-1 antibodies usable in the present disclosure include monoclonal antibodies that bind specifically to human PD-1 and exhibit at least one, in some aspects, at least five, of the preceding characteristics.

Other anti-PD-1 monoclonal antibodies have been described in, for example, U.S. Pat. Nos. 6,808,710, 7,488,802, 8,168,757 and 8,354,509, US Publication No. 2016/0272708, and PCT Publication Nos. WO 2012/145493, WO 2008/156712, WO 2015/112900, WO 2012/145493, WO 2015/112800, WO 2014/206107, WO 2015/35606, WO 2015/085847, WO 2014/179664, WO 2017/020291, WO 2017/020858, WO 2016/197367, WO 2017/024515, WO 2017/025051, WO 2017/123557, WO 2016/106159, WO 2014/194302, WO 2017/040790, WO 2017/133540, WO 2017/132827, WO 2017/024465, WO 2017/025016, WO 2017/106061, WO 2017/19846, WO 2017/024465, WO 2017/025016, WO 2017/132825, and WO 2017/133540 each of which is incorporated by reference in its entirety.

In some aspects, the anti-PD-1 antibody is selected from the group consisting of nivolumab (also known as OPDIVO®, 5C4, BMS-936558, MDX-1106, and ONO-4538), pembrolizumab (Merck; also known as KEYTRUDA®, lambrolizumab, and MK-3475; see WO2008/156712), PDR001 (Novartis; see WO 2015/112900), MEDI-0680 (AstraZeneca; also known as AMP-514; see WO 2012/145493), cemiplimab (Regeneron; also known as REGN-2810; see WO 2015/112800), JS001 (TAIZHOU JUNSHI PHARMA; also known as toripalimab; see Si-Yang Liu et al., J. Hematol. Oncol. 10:136 (2017)), BGB-A317 (Beigene; also known as Tislelizumab; see WO 2015/35606 and US 2015/0079109), INCSHR1210 (Jiangsu Hengrui Medicine; also known as SHR-1210; see WO 2015/085847; Si-Yang Liu et al., J. Hematol. Oncol. 10:136 (2017)), TSR-042 (Tesaro Biopharmaceutical; also known as ANB011; see WO2014/179664), GLS-010 (Wuxi/Harbin Gloria Pharmaceuticals; also known as WBP3055; see Si-Yang Liu et al., J. Hematol. Oncol. 10:136 (2017)), AM-0001 (Armo), STI-1110 (Sorrento Therapeutics; see WO 2014/194302), AGEN2034 (Agenus; see WO 2017/040790), MGA012 (Macrogenics, see WO 2017/19846), BCD-100 (Biocad; Kaplon et al., mAbs 10(2):183-203 (2018), and IBI308 (Innovent; see WO 2017/024465, WO 2017/025016, WO 2017/132825, and WO 2017/133540).

In some aspects, the protein is an anti-PD-L1 antibody. Anti-PD-L1 antibodies that are known in the art can be used in the compositions and methods of the present disclosure. Examples of anti-PD-L1 antibodies useful in the compositions and methods of the present disclosure include the antibodies disclosed in U.S. Pat. No. 9,580,507. Anti-PD-L1 human monoclonal antibodies disclosed in U.S. Pat. No. 9,580,507 have been demonstrated to exhibit one or more of the following characteristics: (a) bind to human PD-L1 with a K_{D} of 1×10⁻⁷ M or less, as determined by surface plasmon resonance using a Biacore biosensor system; (b) increase T-cell proliferation in a Mixed Lymphocyte Reaction (MLR) assay; (c) increase interferon-γ production in an MLR assay; (d) increase IL-2 secretion in an MLR assay; (e) stimulate antibody responses; and (f) reverse the effect of T regulatory cells on T cell effector cells and/or dendritic cells. Anti-PD-L1 antibodies usable in the present disclosure include monoclonal antibodies that bind specifically to human PD-L1 and exhibit at least one, in some aspects, at least five, of the preceding characteristics.

In certain aspects, the anti-PD-L1 antibody is selected from the group consisting of BMS-936559 (also known as 12A4, MDX-1105; see, e.g., U.S. Pat. No. 7,943,743 and WO 2013/173223), atezolizumab (Roche; also known as TECENTRIQ®; MPDL3280A, RG7446; see U.S. Pat. No. 8,217,149; see, also, Herbst et al. (2013) J Clin Oncol 31(suppl):3000), durvalumab (AstraZeneca; also known as IMFINZI™, MEDI-4736; see WO 2011/066389), avelumab (Pfizer; also known as BAVENCIO®, MSB-0010718C; see WO 2013/079174), STI-1014 (Sorrento; see WO2013/181634), CX-072 (Cytomx; see WO2016/149201), KN035 (3D Med/Alphamab; see Zhang et al., Cell Discov. 7:3 (March 2017), LY3300054 (Eli Lilly Co.; see, e.g., WO 2017/034916), BGB-A333 (BeiGene; see Desai et al., KO 36 (15suppl):TPS3113 (2018)), and CK-301 (Checkpoint Therapeutics; see Gorelik et al., AACR:Abstract 4606 (April 2016)).

In some aspects, the protein is an anti-GITR (glucocorticoid-induced tumor necrosis factor receptor family-related gene) antibody. In some aspects, the anti-GITR antibody has the CDR sequences of 6C8, e.g., a humanized antibody having the CDRs of 6C8, as described, e.g., in WO2006/105021; an antibody comprising the CDRs of an anti-GITR antibody described in WO2011/028683; an antibody comprising the CDRs of an anti-GITR antibody described in JP2008278814, an antibody comprising the CDRs of an anti-GITR antibody described in WO2015/031667, WO2015/187835, WO2015/184099, WO2016/054638, WO2016/057841, WO2016/057846, WO 2018/013818, or other anti-GITR antibody described or referred to herein, all of which are incorporated herein in their entireties.

In other aspects, the protein is an anti-LAG3 antibody. Lymphocyte-activation gene 3, also known as LAG-3, is a protein which in humans is encoded by the LAG3 gene. LAG3, which was discovered in 1990 and is a cell surface molecule with diverse biologic effects on T cell function. It is an immune checkpoint receptor and as such is the target of various drug development programs by pharmaceutical companies seeking to develop new treatments for cancer and autoimmune disorders. In soluble form it is also being developed as a cancer drug in its own right. Examples of anti-LAG3 antibodies include, but are not limited to, the antibodies in WO 2017/087901 A2, WO 2016/028672 A1, WO 2017/106129 A1, WO 2017/198741 A1, US 2017/0097333 A1, US 2017/0290914 A1, and US 2017/0267759 A1, all of which are incorporated herein in their entireties.

In some aspects, the protein is an anti-CXCR4 antibody. CXCR4 is a 7 transmembrane protein, coupled to Gl. CXCR4 is widely expressed on cells of hemopoietic origin, and is a major co-receptor with CD4+ for human immunodeficiency virus 1 (HIV-1) See Feng, Y., Broeder, C. C., Kennedy, P. E., and Berger, E. A. (1996) Science 272, 872-877. Examples of anti-CXCR4 antibodies include, but are not limited to, the antibodies in WO 2009/140124 A1, US 2014/0286936 A1, WO 2010/125162 A1, WO 2012/047339 A2, WO 2013/013025 A2, WO 2015/069874 A1, WO 2008/142303 A2, WO 2011/121040 A1, WO 2011/154580 A1, WO 2013/071068 A2, and WO 2012/175576 A1, all of which are incorporated herein in their entireties.

In some aspects, the protein is an anti-CD73 (ecto-5′-nucleotidase) antibody. In some aspects, the anti-CD73 antibody inhibits the formation of adenosine. Degradation of AMP into adenosine results in the generation of an immunosuppressed and pro-angiogenic niche within the tumor microenvironment that promotes the onset and progression of cancer. Examples of anti-CD73 antibodies include, but are not limited to, the antibodies in WO 2017/100670 A1, WO 2018/013611 A1, WO 2017/152085 A1, and WO 2016/075176 A1, all of which are incorporated herein in their entireties.

In some aspects, the protein is an anti-TIGIT (T cell Immunoreceptor with Ig and ITIM domains) antibody. TIGIT is a member of the PVR (poliovirus receptor) family of immunoglobin proteins. TIGIT is expressed on several classes of T cells including follicular B helper T cells (TFH). The protein has been shown to bind PVR with high affinity; this binding is thought to assist interactions between TFH and dendritic cells to regulate T cell dependent B cell responses. Examples of anti-TIGIT antibodies include, but are not limited to, the antibodies in WO 2016/028656 A1, WO 2017/030823 A2, WO 2017/053748 A2, WO 2018/033798 A1, WO 2017/059095 A1, and WO 2016/011264 A1, all of which are incorporated herein by their entireties.

In some aspects, the protein is an anti-OX40 (i.e., CD134) antibody. OX40 is a cytokine of the tumor necrosis factor (TNF) ligand family. OX40 functions in T cell antigen-presenting cell (APC) interactions and mediates adhesion of activated T cells to endothelial cells. Examples of anti-OX40 antibodies include, but are not limited to, WO 2018/031490 A2, WO 2015/153513 A1, WO 2017/021912 A1, WO 2017/050729 A1, WO 2017/096182 A1, WO 2017/134292 A1, WO 2013/038191 A2, WO 2017/096281 A1, WO 2013/028231 A1, WO 2016/057667 A1, WO 2014/148895 A1, WO 2016/200836 A1, WO 2016/100929 A1, WO 2015/153514 A1, WO 2016/002820 A1, and WO 2016/200835 A1, all of which are incorporated herein by their entireties.

In some aspects, the protein is an anti-IL8 antibody. IL-8 is a chemotactic factor that attracts neutrophils, basophils, and T-cells, but not monocytes. It is also involved in neutrophil activation. It is released from several cell types in response to an inflammatory stimulus.

In some aspects, the protein is Abatacept (marketed as ORENCIA®). Abatacept (also abbreviated herein as Aba) is a drug used to treat autoimmune diseases like rheumatoid arthritis, by interfering with the immune activity of T cells. Abatacept is a fusion protein composed of the Fc region of the immunoglobulin IgG1 fused to the extracellular domain of CTLA-4. In order for a T cell to be activated and produce an immune response, an antigen presenting cell must present two signals to the T cell. One of those signals is the major histocompatibility complex (MHC), combined with the antigen, and the other signal is the CD80 or CD86 molecule (also known as B7-1 and B7-2).

In some aspects, the protein is Belatacept (trade name NULOJIX®). Belatacept is a fusion protein composed of the Fc fragment of a human IgG1 immunoglobulin linked to the extracellular domain of CTLA-4, which is a molecule crucial in the regulation of T cell costimulation, selectively blocking the process of T-cell activation. It is intended to provide extended graft and transplant survival while limiting the toxicity generated by standard immune suppressing regimens, such as calcineurin inhibitors. It differs from abatacept (ORENCIA®) by only 2 amino acids.

In some aspects, the protein mixture comprises an antibody, antibody fragment, antigen-binding fragment, a fusion protein, a naturally occurring protein, a chimeric protein, or any combination thereof. In some aspects, the protein mixture comprises an antibody selected from IgM, IgA, IgE, IgD, and IgG. In some aspects, the protein mixture comprises an antibody and the antibody is an IgG antibody selected from IgG1, IgG2, IgG3, and IgG4. In some aspects, the antibody comprises a dual variable domain immunoglobulin. In some aspects, the antibody comprises a trivalent antibody. In some aspects, the antibody or antibody fragment comprises an anti-PD-1, anti-PD-L1 anti-CTLA4, anti-TIM3, anti-LAG3, anti-NKG2a, anti-ICOS, anti-CD137, anti-KIR, anti-TGFβ, anti-IL-10, antiB7-H4, anti-GITR, anti-CXCR4, anti-CD73, anti-TIGIT, anti-OX40, anti-IL-8 antibody or antibody fragment thereof.

In some aspects, the protein mixture comprising the protein of interest is derived from a bacterial, yeast, insect, or mammalian cell culture. In some aspects, the mammalian cell culture is a Chinese hamster ovary (CHO) cell culture.

In some aspects, the protein mixture comprising the protein of interest is obtained from batch cell culture. In some aspects, the protein mixture comprising the protein of interest is obtained from fed-batch cell culture. In some aspects, the protein mixture is produced in a bioreactor. In some aspects, the protein mixture is produced in a single-use bioreactor. In some aspects the protein mixture is obtained from perfusion cell culture. In some aspects, the protein mixture is produced in a perfusion of TFF perfusion bioreactor. In some aspects, the protein mixture is produced in a cell culture lasting from about 1 to about 60 days. In some aspects, the protein mixture is produced in a cell culture lasting about 25 days.

### IV. Pharmaceutical Compositions

The proteins produced by the methods of the present disclosure can be further formulated to be suitable for human administration, e.g., pharmaceutical composition. A composition that is acceptable for pharmaceutical administration, such a composition can include substances that are impurities at a level not exceeding an acceptable level for pharmaceutical administration (such level including an absence of such impurities), and can include pharmaceutically acceptable excipients, vehicles, carriers and other inactive ingredients, for example, to formulate such composition for ease of administration, in addition to any active agent(s). The compositions prepared by the methods of the present disclosure are useful to treat a variety of diseases.

### V. Ultrafiltration Systems

The present disclosure provides systems for reducing a filtration process time of a protein of interest. The present disclosure is also provides systems for concentrating a protein of interest.

In some aspects, a system for concentrating a protein of interest, comprises: a feed tank, a reservoir tank connected to the feed tank by a first fluid pathway; a filtration membrane connected to the reservoir tank by a second fluid pathway; and a three-way valve, wherein the three-way valve is connected to the filtration membrane by a third fluid pathway, wherein the three-way valve is connected to the reservoir tank by a fourth fluid pathway, and wherein the three-way valve is connected to the feed tank by a fifth fluid pathway, wherein the reservoir tank receives a protein mixture comprising the protein of interest from the feed tank via the first fluid pathway, wherein the filtration membrane receives the protein mixture comprising the protein of interest from the reservoir tank via the second fluid pathway and filters the protein mixture, and wherein the three-way valve receives retentate from the filter via the third fluid pathway and directs the retentate either to the reservoir tank via the fourth fluid pathway or to the feed tank via the fifth fluid pathway.

In some aspects, the three-way valve directs the retentate to the reservoir tank if the total volume of the protein mixture within the system is less than the capacity of the reservoir tank, and wherein the three-way valve directs the retentate to the feed tank if the total volume of the protein mixture within the system is greater than the capacity of the reservoir tank.

In some aspects, the system further comprises a sensor configured to determine the total volume, weight, and/or concentration of the protein mixture within the system, wherein the three-way valve automatically directs the retentate either to the reservoir tank or to the feed tank based on feedback from the sensor. In some aspects, an in-line UV-visible spectrophotometer is used to monitor the protein concentration in the protein mixture in real time. In some aspects, level sensors is used to monitor the volume of the protein solution in either or both the feed tank and reservoir tank in real time. In some aspects, these level sensors include, but are not limited to, guided wave radar or membrane-based pressure level sensors. In some aspects, the volume of protein solution in either or both the feed tank and reservoir tank is monitored gravimetrically, where the mass of the protein solution in the feed and/or reservoir tanks is measured using a scale, and the mass is converted to a volume using the solution density. In some aspects, the system further comprises one or more diaphram pumps, rotary lobe pumps, or peristaltic pumps.

In some aspects, the filter comprises an in-line filtration membrane. In some aspects, the in-line filtration membrane is an ultrafiltration membrane. In some aspects, the in-line filtration membrane is. In some aspects, the in-line filtration membrane is polyvinylether, polyvinylalcohol, nylon, silicon, polysilicon, ultrananocrystalline diamond, diamond-like-carbon, silicon dioxide, titanium, silica, silicon nitride, polytetrafluorethylene, silicone, polymethacrylate, polymethyl methacrylate, polyacrylate, polystyrene, polyacrylamide, polymethacrylamide, polycarbonate, graphene, graphene oxide, polysaccharides, ceramic particles, poly(styrenedivinyl)benzene, polysulfone, polyethersulfone, modified polyethersulfone, polyarylsulfone, polyphenyl sulfphone, polyvinyl chloride, polypropylene, cellulose acetate, cellulose nitrate, polylactic acid, polyacrylonitrile, polyvinylidene fluoride, polypiperazine, polyamide-polyether block polymers, polyimide, polyetherimide, polyamide, regenerated cellulose, composite regenerated cellulose, or combinations thereof. In some aspects, the in-line filtration membrane is polyethersulfone. In some aspects, the in-line filtration membrane is cellulose. In some aspects, the in-line filtration membrane is a combination of polyethersulfone and cellulose. In some aspects, filtration membrane has a molecular weight cutoff (MWCO) lower than from about 50 kD to about 5 kD. In some aspects, the filtration membrane has a MWCO lower than about 5 kD.

### EXAMPLES

### Example 1

The fed-batch process time is a function of multiple facility fit parameters, namely the retentate vessel volume, the system holdup volume, the load protein concentration and load volume.

When the load material volume is too large for the load to fit within the retentate vessel, part of the load material is kept in the feed tank and slowly fed into the retentate vessel during fed-batch ultrafiltration. The point at which the retentate volume has decreased sufficiently to contain the entire protein load (in grams of protein) in the retentate vessel is referred to as the crossover concentration (Eqn. 1):

$${c}_{crossover} = \frac{{c}_{{0}^{* {V}_{0}}}}{{V}_{r e s} + {V}_{holdup}} \qquad ( 1 )$$

where C₀ and V₀ are the protein load concentration and volume respectively, Vᵣₑₛ is the retentate vessel volume and V_{holdup} is the system holdup volume.

The process time required to concentrate the protein in fed-batch mode can be calculated numerically using Eqn. 2c, which was derived by integrating the flux equation (Eqn. 2a, defined with respect to the cumulative permeate volume V′) and assuming the concentration polarization model for flux (Eqn. 2b). The limit of integration V_{perm,final} in Eqn. 2c is the expected permeate volume at the crossover concentration. The protein concentration in fed-batch operation can be defined as a function of the cumulative permeate volume V′ by Eqn. 3, where Vᵣₑₛ is the filled volume of the retentate vessel, and which is kept constant during fed-batch loading. V_{perm,final} can therefore be calculated by solving Eqn. 3 for V′ using the crossover protein concentration (Eqn. 1).

$$\frac{d V ′}{d t} = J ( c ) * {A}_{m} \qquad ( 2 a )$$

$$J ( c ) = {k}_{c} * ln ( \frac{{c}_{w}}{c} ) \qquad ( 2 b )$$

$${\int }_{0}^{t} d t = {\int }_{0}^{{V}_{p e rm , final}} \frac{1}{J ( c ) * {A}_{m}} {dV}^{′} \qquad ( 2 c )$$

$$c ( {V}^{′} ) = \frac{{c}_{0} * ( {V}_{r e s} + {V}^{′} )}{{V}_{r e s} + {V}_{holdup}} \qquad ( 3 )$$

From these equations, it is clear that the fed-batch process time is a function of multiple facility fit parameters, namely the retentate vessel volume, the system holdup volume, the load protein concentration and load volume.

### Example 2

The traditional fed-batch TFF configuration is illustrated in FIG. 1A. Here, the feed tank holding the load material is connected to the retentate vessel (e.g., reservoir) by a feed pump, and the retentate vessel is separately incorporated into a recirculation loop with the TFF membrane device and a recirculation pump.

In the pseudo-batch configuration described herein, the flow path is modified to incorporate the feed tank into the recirculation loop, as illustrated in FIG. 1B. A three-way valve is placed after the retentate port of the TFF filter module to direct the retentate flow to either the feed tank or retentate vessel depending on the status of the first ultrafiltration step, as will be explained below. Mixers (not shown) are used for both tanks/vessels.

During the initial loading stage of the first ultrafiltration step, the three-way valve is set to direct the retentate flow to the load tank and block off flow to the retentate vessel. The load material is fed from the feed tank into the retentate vessel with the feed pump as normal, but the retentate from the TFF filter module is returned to the feed tank and mixed with the remaining load material instead. The new load material is now slightly more concentrated, and fed to the retentate vessel, and cycle repeats. The protein solution in both the load tank and retentate vessel therefore are concentrated at the same rate, unlike in fed-batch loading where the retentate becomes increasingly more concentrated while the load material remains fixed at the initial dilute concentration. With this configuration, the feed tank effectively acts as an extension of the retentate vessel, and the two act as a single reservoir in a recirculation loop, akin to a batch setup (FIG. 1C). The pseudo-batch configuration therefore converts the otherwise hybrid-mode process to a batch-like process, mitigating the time penalty for the fed-batch loading portion of the first ultrafiltration step as well as the facility fit dependency of the process time.

When the loading step is complete (e.g., when the total protein solution volume has decreased to below the retentate vessel volume), the three-way valve is actuated to re-direct the retentate flow to the retentate vessel and block flow to the feed tank, effectively eliminating the feed tank from the recirculation loop and converting the TFF setup to a true batch configuration. The feed pump can be operated for a little longer to perform an air chase (into the retentate vessel) of the residual material in the connecting tubing between the two vessels in order to maximize recovery of the load material.

As a proof of concept to demonstrate the advantages of the pseudo-batch configuration described above, high concentration solutions (˜180 g/L) of mAb A were generated by TFF performed at lab scale in the batch, hybrid and pseudo-batch configurations. The process times for each filtration step (first ultrafiltration (UF1), diafiltration (DF) and second ultrafiltration (UF2)) as well as the quality attributes (e.g., high molecular weight (HMW) species, particle counts) of the post-recovery purified drug substance (PDS) were compared between the three configurations. The HMW levels were measured by high performance liquid size exclusion chromatography using an Alliance 2695 HPLC system equipped with a Model 2487 dual wavelength detector (Waters Corporation, Milford MA USA) and TSKgel SuperSW3000 main and guard columns (Tosoh Bioscience, King of Prussia PA USA), while the particle counts for particulates between 1 and 100 μm were quantified by micro-flow imaging using the MFI 5200 (Protein Simple, San Jose CA USA). The PDS turbidity was measured in triplicate using a Hach 2100Q turbidimeter, which was calibrated daily before use.

The TFF experiments were performed using a PendoTECH control and data acquisition system (PendoTECH, Princeton NJ USA) equipped with a Quattroflow 150 pumps (High Purity New England, Smithfield RI USA) and 88 cm² 30 kDa Ultracel Pellicon 3 D-screen membranes (MilliporeSigma, Burlington MA USA). A single batch of purified 10 g/L mAb solution was split into separate aliquots to generate identical load materials for the three runs, where the aliquot volume was defined to achieve a membrane loading of approximately 600 g/m². For both the pseudo-batch and hybrid configurations, the volume ratio of the initial load volume to retentate vessel plus system holdup volume was set to 5. For each run, the mAb was concentrated to 50 g/L during the first ultrafiltration step and then buffer exchanged with 5 diavolumes of the diafiltration buffer. The diafiltered protein solution was then further concentrated to 180 g/L (determined gravimetrically from the retentate and permeate masses, and accounting for the change in solution density at high concentration) in a second ultrafiltration step. The drug substance was then recovered by chasing the residual protein solution out of the holdup volume with buffer, where the volume of chase buffer used was 1.2 times the system holdup volume.

### Example 3

### A. Process Time

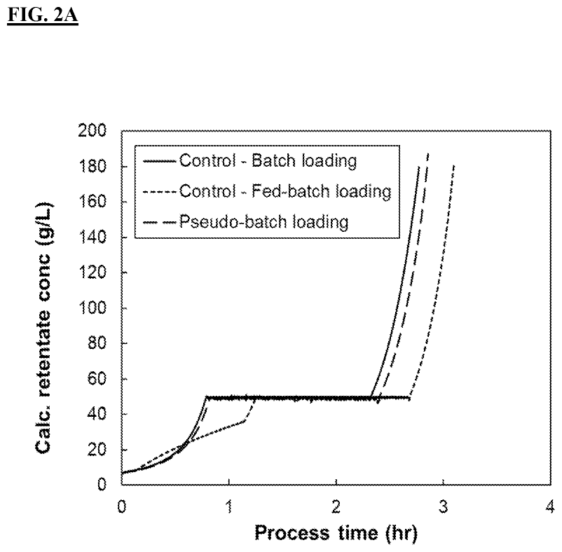

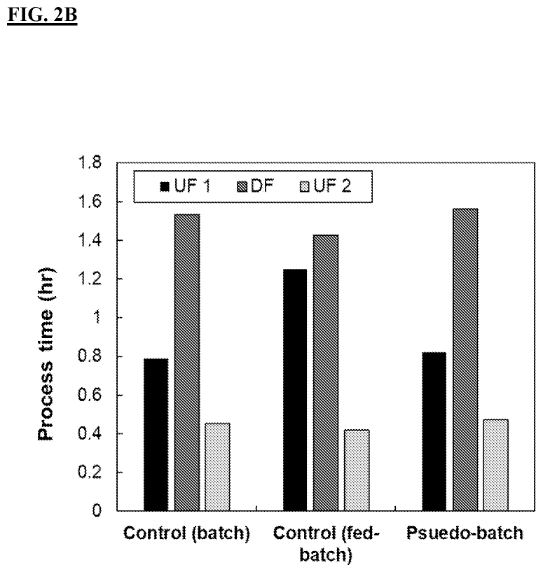

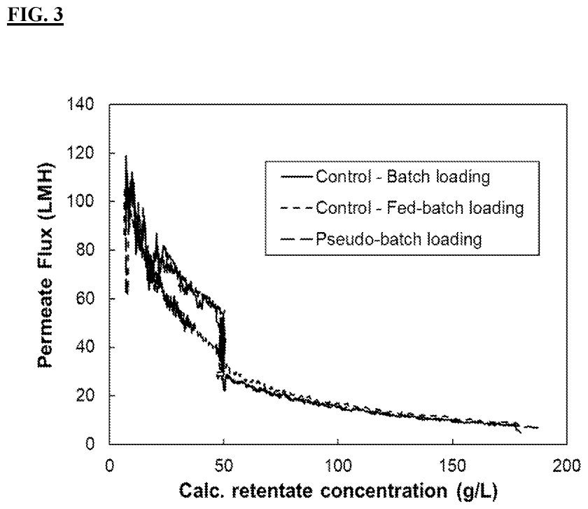

The approximate retentate mAb concentration (calculated from the retentate volume and total mass of protein in the system) as a function of process time is shown for the three configurations in FIG. 2A. The corresponding process times for each stage of the process (UF1, DF, and UF2) are shown in FIG. 2B. The corresponding permeate fluxes observed for the three runs are shown in FIG. 3.

The process time for the UF1 step of the hybrid (fed-batch loading) run was 60% longer than for the batch run, as seen in FIG. 2B. The permeate flux during the loading step (initial part of UF1) was observed to be lower using the fed-batch strategy (FIG. 3), which likely also contributed to the longer UF1 process time of the hybrid run. In contrast, both the UF1 process time (FIG. 2B) and permeate flux (FIG. 3) for the pseudo-batch run were nearly identical to the batch run. Unsurprisingly, the diafiltration and UF2 process times were nearly identical between all three runs (FIG. 2B), since the DF and UF2 steps are necessarily performed in the batch configuration, irrespective of the system configuration during the loading (UF1) step. The small differences in the diafiltration process time between the three runs was due to mild variability in the protein concentration during the diafiltration step (49-51 g/L). These results illustrate the impact of the system configuration during the initial sample loading (UF1) step on the total process time, and demonstrates the ability of the pseudo-batch configuration to mitigate the time penalty associated with fed-batch loading during the UF1 step, as well as eliminate the process time scalability challenges associated with hybrid (fed-batch loading) processes.

### B. Quality Attributes

The impact of the pseudo-batch configuration on drug substance quality attributes was evaluated to determine whether the incorporation of the second pump (e.g., the feed pump) into the recirculation loop would have an adverse effect, as the protein solution now undergoes two pump passes instead of one in every recirculation cycle.

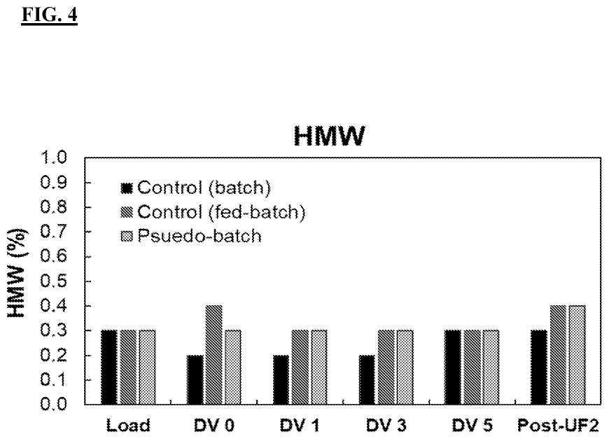

mAb A was found to be insensitive to pump shear exposure with respect to the formation of soluble high molecular weight (HMW) species during UF/DF. The HMW levels remained essentially unchanged throughout the entire process for all three configuration strategies (FIG. 4). The increased pump shear exposure associated with the pseudo-batch configuration therefore did not appear to have an adverse effect on HMW formation.

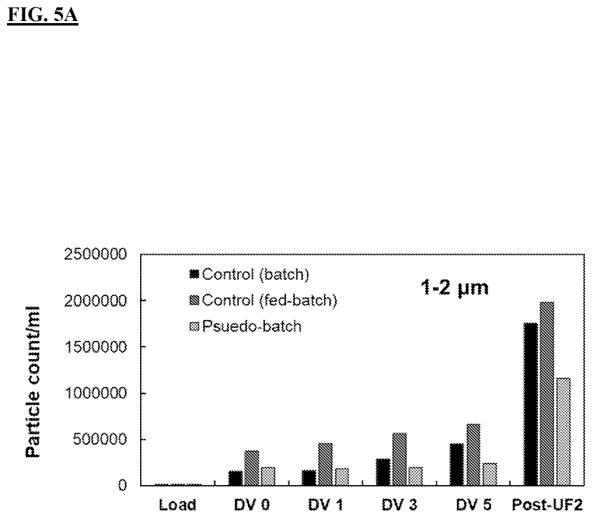

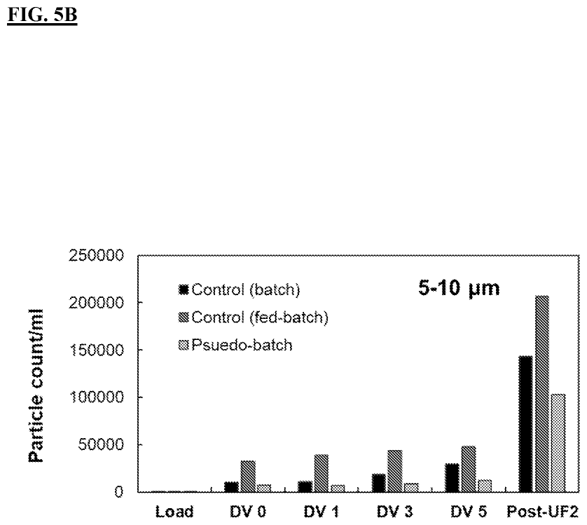

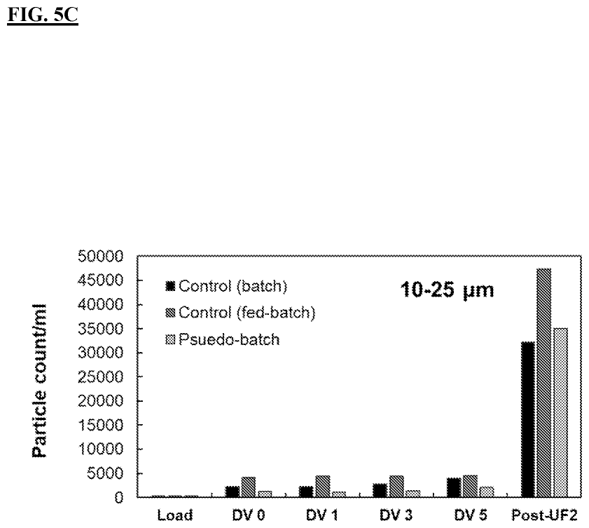

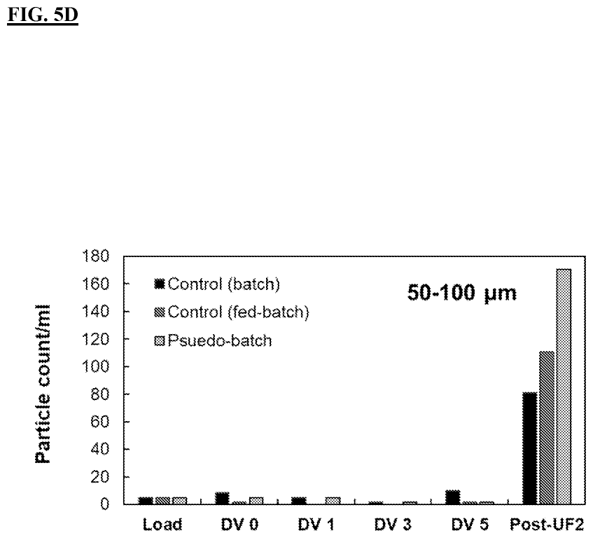

The impact of the pseudo-batch configuration on the larger, insoluble aggregates was quantified by microfluidic imaging (MFI). The subvisible (1-100 μm) particle counts for the in-process pools generated using the three configurations are shown in FIGS. 5A-D. Unlike what was observed for HMW formation, mAb A did show a significant increase in subvisible particle formation over time, where both the total number of particles and the relative particle size distribution differs noticeably between configurations.

The hybrid run consistently had higher particle counts than the batch run for all particle size ranges, consistent with the longer process time and corresponding increased exposure of the protein to shear and interfacial stresses. In contrast, the pseudo-batch run generated comparable or lower amounts of particles than the batch run in the 1-25 μm size range (FIGS. 5A-C), with the exception of the 50-100 μm size range (FIG. 5D). The higher particle count for the 50-100 μm size range may potentially be caused by an increased level of (undetected) aggregate precursors generated during the UF1 step as a result of the double pump passes compared to the other two loading strategies. However, it is important to note that the total particle count for the 50-100 μm size range is several orders of magnitude smaller than the particle counts for the smaller particle size ranges. From the perspective of overall particle generation, the pseudo-batch configuration is a noticeable improvement over the fed-batch configuration, and appears to generate drug substance of comparable quality to the batch configuration.

It is interesting to note that the biggest difference in particle counts between the three configurations occurs between DVS and post-UF2, despite the fact that the UF2 step is the shortest part of the overall process (FIG. 2B). Given that all three configurations have the same process time and setup for both the DF and UF2 step, these results demonstrate the importance of the UF1 step in generating aggregation precursors that may not be detected by SEC/MFI, but which impact the quality of the final drug substance.

### Example 4

To determine if the type of feed pump affects the pseudo-batch process, the diaphragm feed pump was replaced with a peristaltic feed pump. A ˜180 g/L solution of mAb A was generated using the pseudo-batch loading configuration with the same process parameters as the pseudo-batch run in Example 3 (e.g., membrane loading, load concentration, diafiltration concentration, diavolumes exchanged, pump feed flux, and TWIT.). The volume ratio of the initial load volume to the retentate tank volume was kept at 5 (i.e. the liquid volume in the retentate tank during the loading step was maintained at a constant value equal to 20% of the initial total load volume), as in Example 3. The process performance of this run was compared to that for the pseudo-batch run using the diaphragm feed pump.

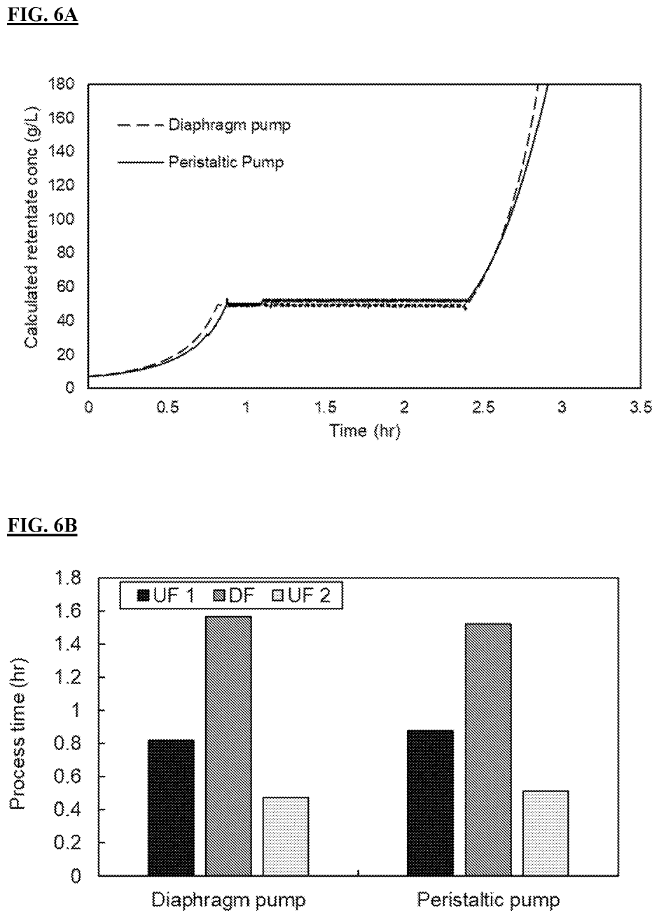

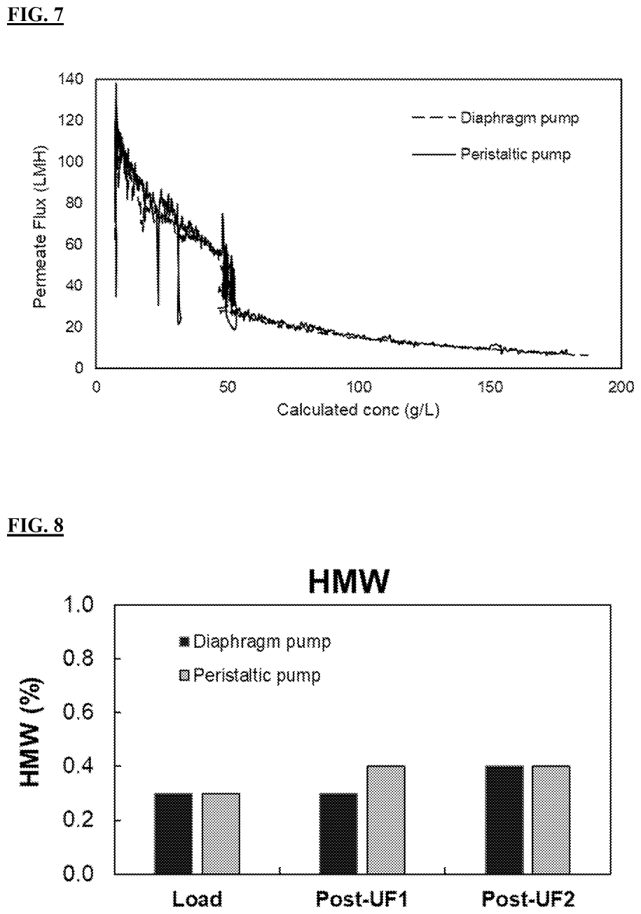

The approximate retentate mAb concentration (calculated from the retentate volume and total mass of protein in the system) as a function of process time is shown for the two pseudo-batch runs in FIG. 6A. The corresponding process times for each stage of the process (UF1, DF and UF2) are shown in FIG. 6B. The permeate flux as a function of calculated retentate concentration are shown in FIG. 7. The flux profiles as well as process times for the two runs are nearly identical for each stage of the UF/DF process, indicating that the choice of a peristaltic or diaphragm pump for the feed pump has no impact on the performance of the pseudo-batch loading method with regards to the process throughput.

The drug substance quality attributes were also evaluated to determine whether the feed pump type in the pseudo-batch configuration would significantly impact protein stability during the UF/DF process, which could be a deciding factor in the general practicality of the pseudo-batch method. As can be seen from FIG. 8, the use of the peristaltic feed pump did not cause any increase in HMW formation during UF/DF (within assay variability) over the diaphragm feed pump. The differences in shear exposure associated with the use of a peristaltic feed pump over a diaphragm feed pump therefore did not appear to have an adverse effect on HMW formation.

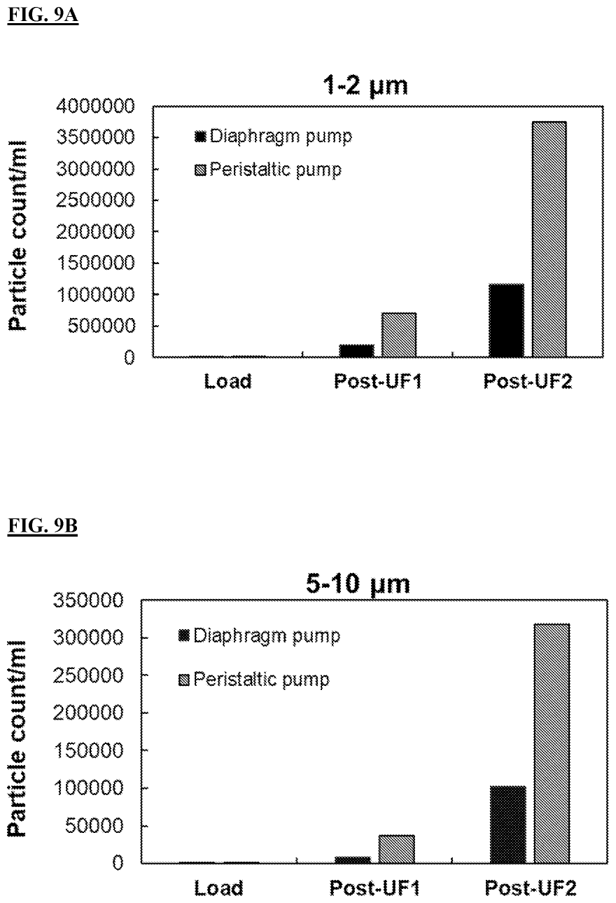

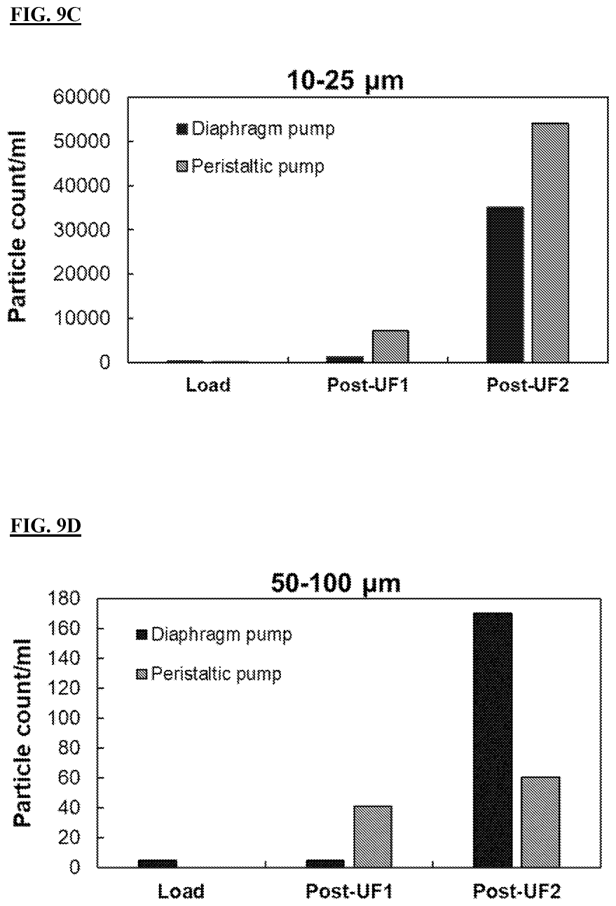

The impact of the feed pump type on the larger, insoluble aggregates was quantified by microfluidic imaging (MFI). The subvisible (1-100 μm) particle counts for the in-process pools generated using the feed pump types are shown in FIGS. 9A-9D. Unlike what was observed for HMW formation, both the total number of particles and the relative particle size distribution differed noticeably between pump types. The peristaltic feed pump led to the generation of significantly more smaller particles (<25 μm) but fewer large particles (>50 μm) compared to the diaphragm pump. This profile suggests that the peristaltic pump generates a higher shear or more turbulent flow regime as the protein solution circulates through the feed pump, which destabilizes larger particulates and results in a relatively higher population of smaller particulates. However, the differences in particulate size distribution do not appear to impact the membrane flux or process throughput, as described above. As these particulates are removed during the final formulation and filtration step after the UF/DF step, the difference in particulate generation between the two pump types is therefore unlikely to be a concern for the final product quality.

### Example 5

To determine whether the retentate to total load volume ratio during the loading step would impact the pseudo-batch performance, the liquid volume in the retentate tank during the loading step was kept constant at a lower volume relative to the total load volume. A ˜180 g/L solution of mAb A was generated using the pseudo-batch loading method using the UF/DF process parameters and configuration (peristaltic feed pump) described in Example 4. However, the liquid volume in the retentate tank was kept at 10% of the total initial load volume during the loading part of the UF1 step, instead of 20% as in Example 4. The process performance of this run was compared to that for the pseudo-batch run where the retentate tank volume was maintained at 20% of the total initial load volume during the loading step.

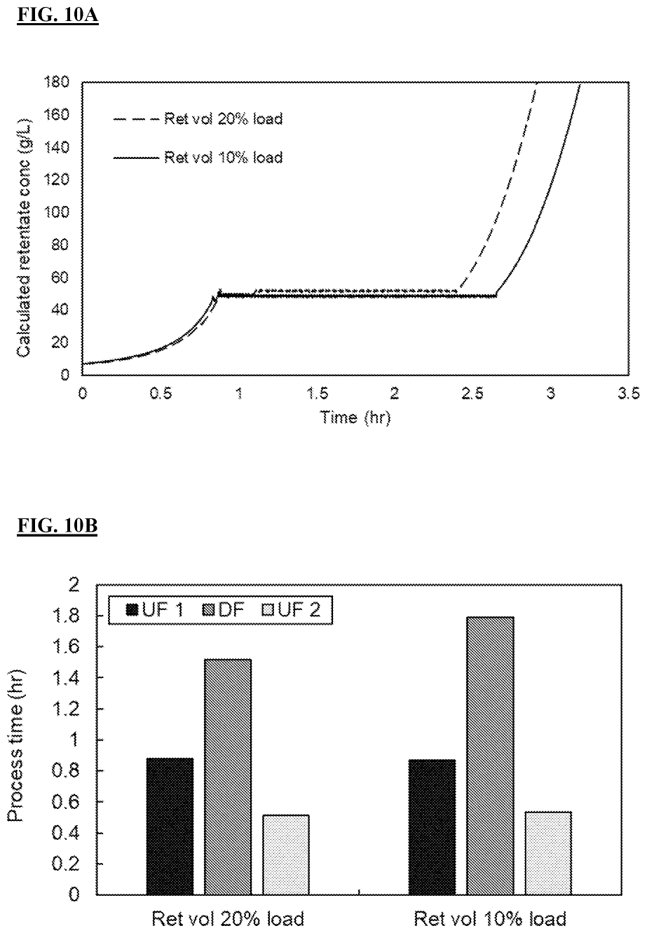

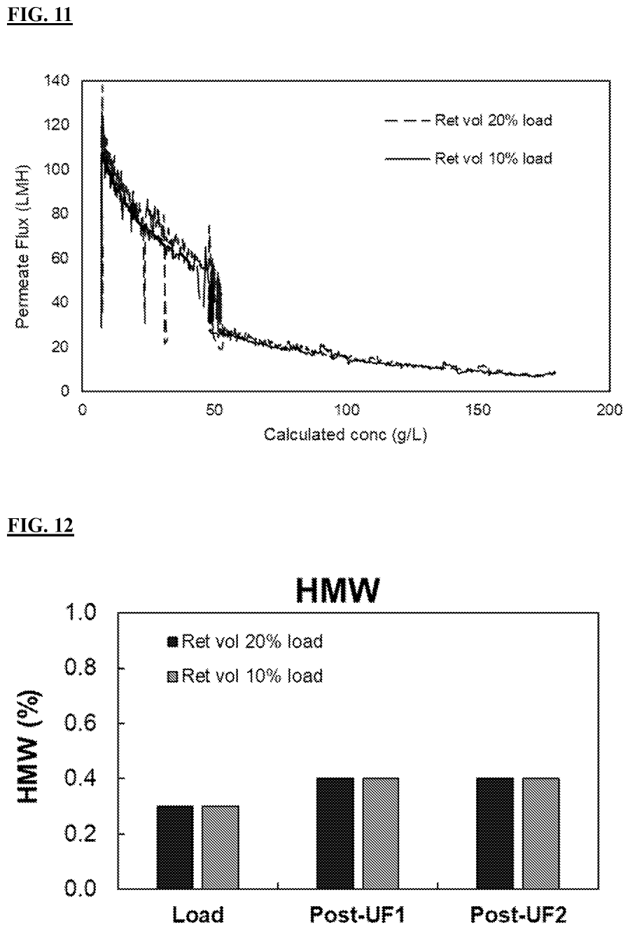

The approximate retentate mAb concentration (calculated from the retentate volume and total mass of protein in the system) as a function of process time is shown for the two pseudo-batch runs in FIG. 10A. The corresponding process times for each stage of the process (UF1, DF and UF2) are shown in FIG. 10B. The permeate flux as a function of calculated retentate concentration are shown in FIG. 11. The flux profiles as well as UF1 and UF2 process times for the two runs are nearly identical, indicating that the retentate tank to total load volume ratio during the loading step has no impact on the process time required to concentrate the protein. The small difference in diafiltration time is likely due to slight variation in the actual diafiltration concentration around the target value of 50 g/L. This result is unlike for fed-batch operation, where the UF1 process time depends on the relative volume ratio of the retentate tank and total load volume, as explained previously in Example 1. The independence of the UF1 process time on the relative retentate and load volume ratios during the loading step in Examples 4 and 5 is consistent with the governing principle of the pseudo-batch method, wherein the linking of the feed tank and retentate tank in the UF/DF recirculation loop allows them to function effectively as a single reservoir and convert the loading step into a batch process.

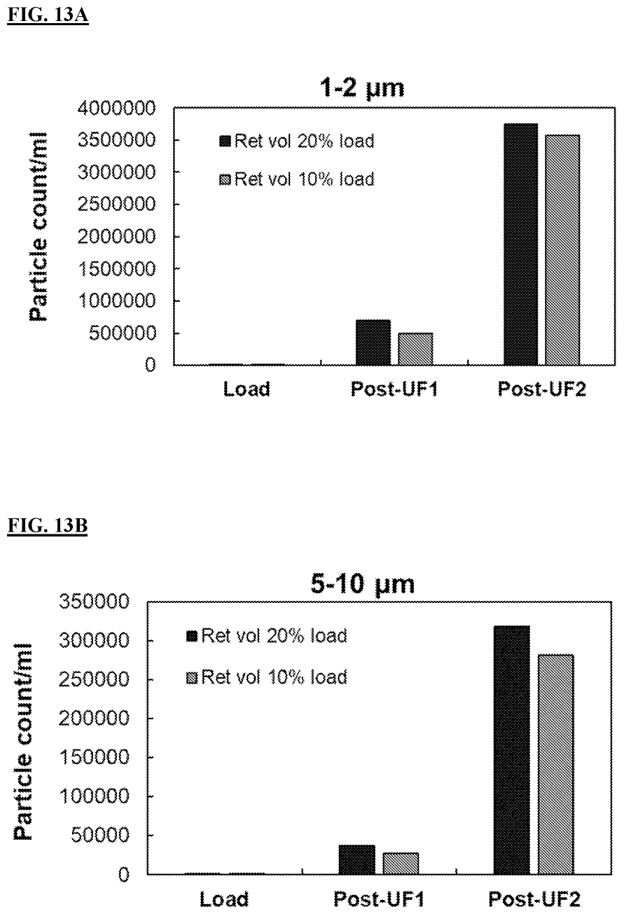

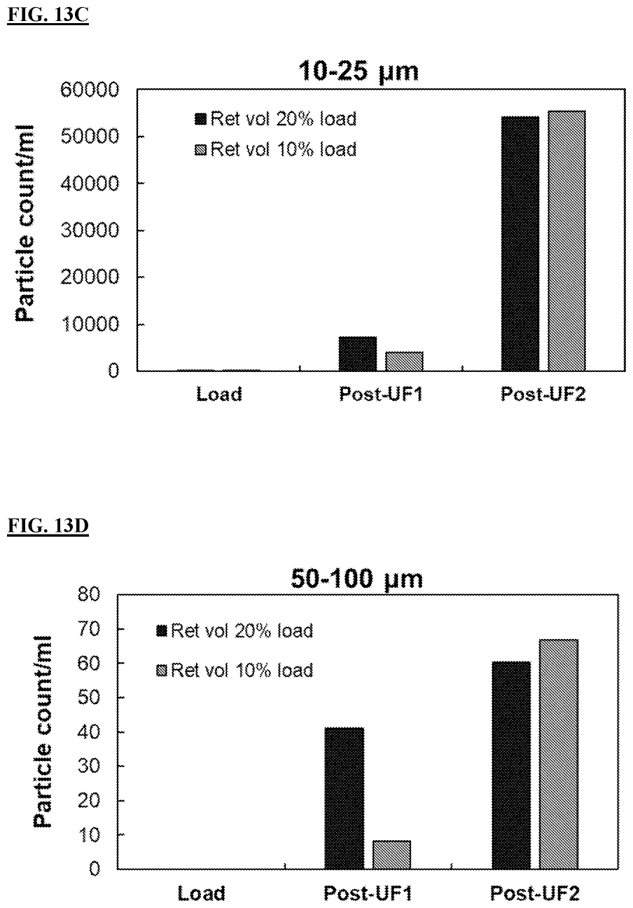

The drug substance quality attributes were also evaluated to determine whether the retentate to total load volume ratio during the loading step would impact protein stability during the UF/DF process. As can be seen from FIG. 12, the run performed where the retentate volume was maintained at 10% of the initial load volume had the same HMW level as the run performed with a volume ratio of 20%. Similarly, the particle size distribution (from 1-100 μm) of the post-UF2 solution as quantified by MFI were nearly identical between the two runs, as seen in FIG. 13A through FIG. 13D. The similarity of the HMW and large particle profiles between the two runs may be attributed in part to the identical UF1 process times (FIG. 10B) and concentration profiles as a function of process time (FIG. 10A), as the protein undergoes the same number of pump passes and other stress factors between the two runs as a result.

### Example 6

The pseudo-batch method was performed on a non-mAb therapeutic protein (MW=˜20 Da), which was used as the load material. The protein was concentrated from 0.7 to 15.5 g/L using the traditional fed-batch as well as the pseudo-batch methods. No buffer exchange was performed during these two runs. All other process parameters (membrane loading, pump feed flux, TMP) were held constant between the two runs. The liquid volume in the retentate tank was kept constant at a value equal to 30% of the total initial load volume throughout the loading step for both runs, and a peristaltic pump was used as the feed pump.

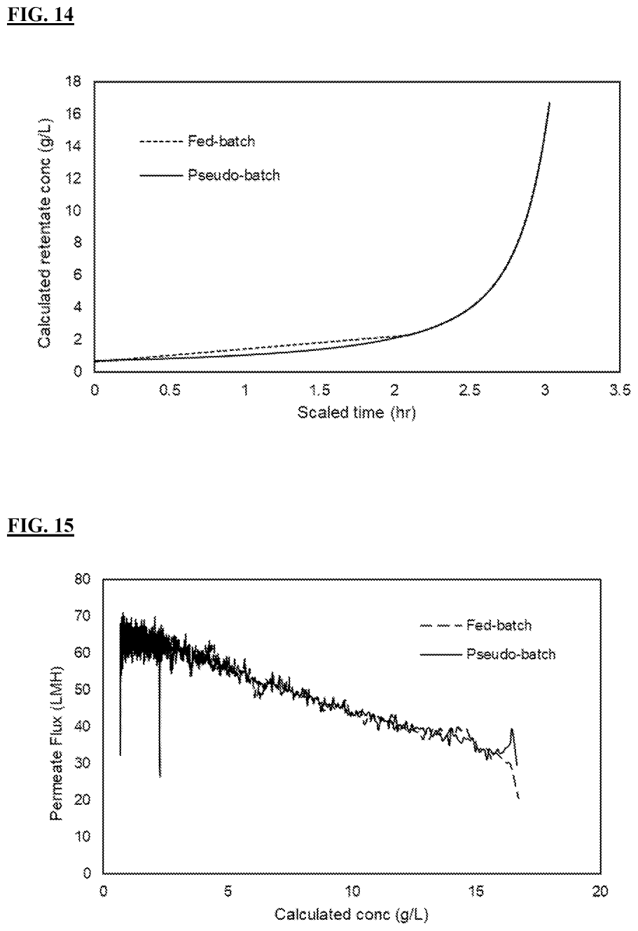

The approximate retentate protein concentration (calculated from the retentate volume and total mass of protein in the system) as a function of process time is shown for the two runs in FIG. 14. The corresponding process times were 3.0 hours for the fed-batch run and 2.9 hours for the pseudo-batch run. The permeate flux as a function of calculated retentate concentration are shown in FIG. 15. The flux versus concentration profiles are identical, indicating that the differences in the loading method do not impact the inherent membrane performance, consistent with observations from the prior Examples. In this example, the loading step of the process occurs over a very small and dilute concentration range (0.7-2 g/L), such that flux decay across this concentration range is essentially negligible, resulting in nearly identical average permeate fluxes during the loading step and consequently similar process times between the two runs. However, the pseudo-batch loading strategy still offers the advantage of reducing the process time compared to the fed-batch loading method, although the difference in this case is very small (˜0.1 hours) due to the ultralow concentration range over which the loading step occurs.

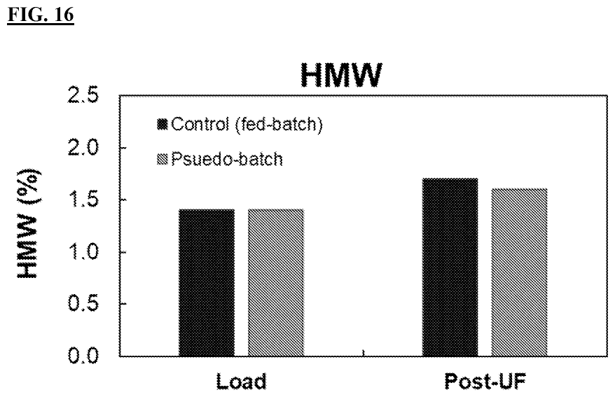

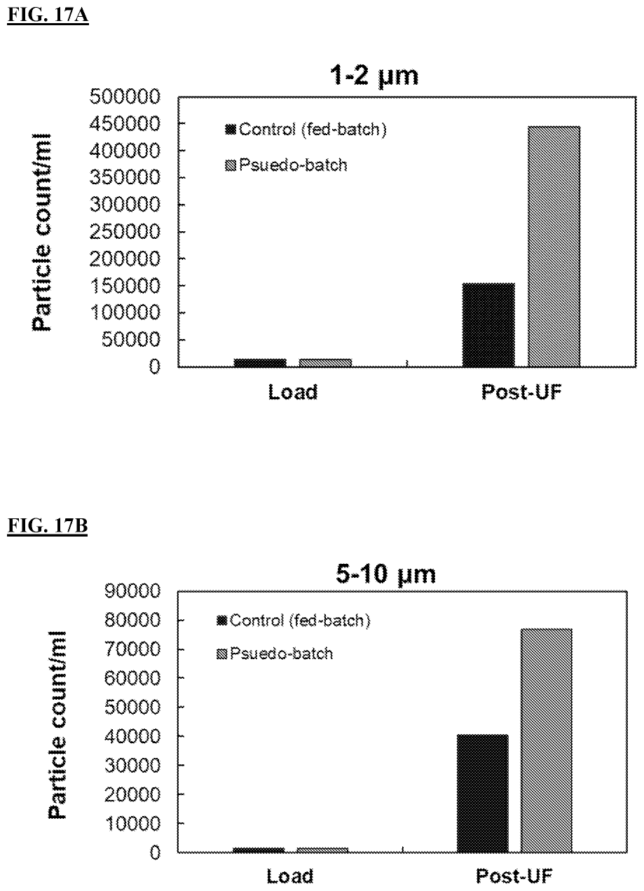

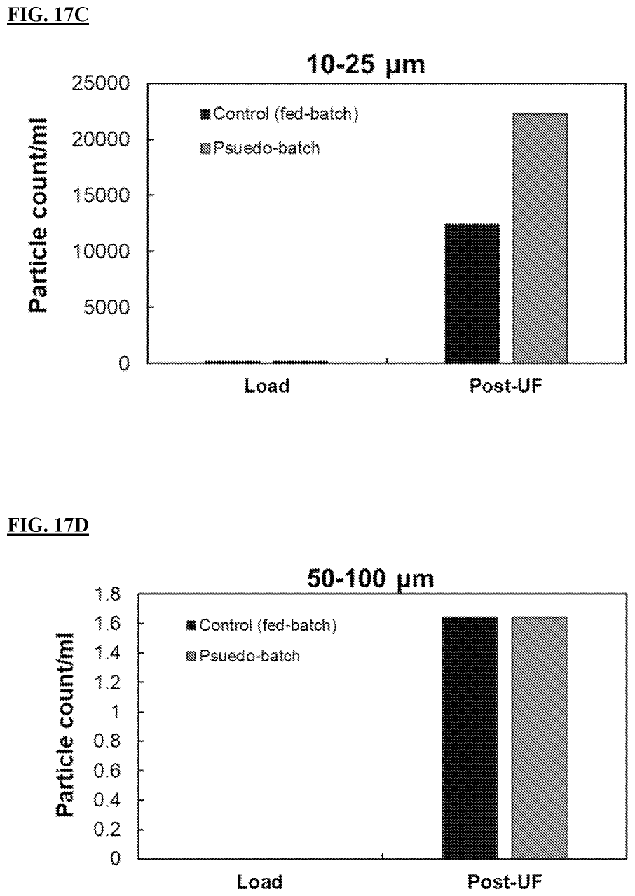

The drug substance quality attributes were also evaluated to determine whether the pseudo-batch loading strategy would have an adverse impact on the stability of the non-mAb protein during UF/DF as a result of the increased number of pump passes that the protein experiences. As can be seen from FIG. 16, the pseudo-batch and fed-batch methods generated comparable amounts of HMW species. However, the two methods differed in the amount and relative size distribution of larger particles formed during UF/DF, as characterized by WI. As can be seen in FIG. 17A through FIG. 17D, the pseudo-batch loading method generates more small particles (<25 μm) but comparable amounts of larger particles (>50 μm) as the fed-batch method. This result is consistent with that seen for mAb A in Examples 3 through 5. The extra number of pump passes inherent to the pseudo-batch loading method causes an increase in the formation of smaller particulates, but not on larger particulates. As the manufacturing bioprocess typically contains a final formulation and filtration step after the UF/DF step, these particulates are expected to be removed from the final drug substance. As such, the increased formation of small particles caused by the pseudo-batch method is not expected to have an adverse impact on the quality of the final drug product.

The pseudo-batch configuration for UF/DF described herein is able to mitigate the time penalty associated with fed-batch loading by converting the fed-batch setup (using a feed tank and retentate vessel) into a batch-like operation (connecting the two vessels in such a way that they act as a single vessel in the TFF recirculation loop). This conversion also eliminates the scale-dependency of the process time that is associated with hybrid processes (e.g., fed-batch loading+batch concentration), making the process fully scalable. In addition, the inclusion of the feed pump into the recirculation loop, resulting in twice as many pump passes compared to a batch configuration, did not cause any noticeable adverse effects on product quality as quantified by HMW formation and subvisible particle counts.

Hybrid UF/DF processes will become necessarily more prevalent for generating high concentration drug substances as the biopharmaceutical industry increasingly moves towards subcutaneous formats for drug delivery. The pseudo-batch configuration described herein can be used instead to reduce the time penalty associated with fed-batch loading, eliminate the process time's scale dependency that results from the hybrid process, and potentially improve upon the product quality compared to the hybrid process. Taken together, these benefits can improve process throughput and yield, as well as improve the scalability of the UF/DF process for more streamlined technology transfer from the lab/development scale to the manufacturing scale.

The foregoing description of the specific aspects will so fully reveal the general nature of the invention that others can, by applying knowledge within the skill of the art, readily modify and/or adapt for various applications such specific aspects, without undue experimentation, without departing from the general concept of the present invention. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

All publications, patents, and patent applications mentioned in this specification are herein incorporated by reference to the same extent as if each individual publication, patent, or patent application was specifically and individually indicated to be incorporated by reference.

Other aspects of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of reducing a filtration process time of a protein of interest, comprising continuously loading a feed tank with a protein mixture comprising the protein of interest that has been filtered at least once (“retentate”), wherein the feed tank is separate from a main reservoir (“retentate”) tank.

2. A method of concentrating a protein of interest comprising continuously loading a feed tank with a protein mixture comprising the protein of interest that has been filtered at least once (“retentate”), wherein the feed tank is separate from a main reservoir (“retentate”) tank.

3. The method of claim 1 or 2, wherein the feed tank further comprises an initial protein mixture comprising a protein of interest that has not been filtered at least once.

4. The method of claim 3, wherein the initial protein mixture and the retentate are mixed together.

5. The method of claim 3 or 4, wherein the protein mixture and/or the retentate are filtered through a filter.

6. The method of claim 5, wherein the filtered protein mixture and the retentate (“retentate”) are loaded into the feed tank.

7. The method of any one of claims 1 to 6, wherein the loading is continued until the protein of interest is concentrated at least about 1 mg/mL, at least about 10 mg/mL, at least about 20 mg/mL, at least about 30 mg/mL, at least about 40 mg/mL, at least about 50 mg/mL, at least about 60 mg/mL, at least about 70 mg/mL, or at least about 80 mg/mL.

8. The method of any one of claims 1 to 7, wherein the loading is continued until the protein of interest is concentrated between about 1 mg/mL and 80 mg/mL, about 5 mg/mL and 70 mg/mL, about 10 mg/mL and 60 mg/mL, about 10 mg/mL and 50 mg/mL, about 10 mg/mL and 40 mg/mL, about 10 mg/mL and 30 mg/mL, about 10 mg/mL and 20 mg/mL, about 20 mg/mL and 70 mg/mL, about 20 mg/mL and 60 mg/mL, about 20 mg/mL and 50 mg/mL, about 20 mg/mL and 40 mg/mL, or about 20 mg/mL and 30 mg/mL.

9. The method of any one of claims 1 to 8, wherein the loading of the retentate is repeated at least twice, at least three times, at least four times, at least five times, at least six times, at least seven times, at least eight times, at least nine times, at least 10 times, at least 20 times, at least 30 times, at least 40 times, at least 50 times, at least 60 times, at least 70 times, at least 80 times, at least 90 times, at least 100 times, at least 110 times, at least 120 times, at least 130 times, at least 140 times, at least 150 times, at least 160 times, at least 170 times, at least 180 times, at least 190 times, at least 200 times, at least 210 times, at least 220 times, at least 230 times, at least 240 times, at least 250 times, at least 260 times, at least 270 times, at least 280 times, at least 290 times, or at least 300 times.

10. The method of any one of claims 1 to 9, further comprising stopping the loading of the retentate to the feed tank.

11. The method of claim 10, further comprising directing the retentate to a reservoir tank.

12. A method of reducing a filtration process time of a protein of interest, comprising loading a protein mixture which comprises the protein of interest to a filtration system comprising a feed tank, a reservoir tank, a filter, a three way valve comprising a feed tank valve connecting the filter to the feed tank and the reservoir tank valve connecting the filter to the reservoir tank, and a reservoir input connecting the feed tank and the reservoir tank.

13. A method of concentrating a protein of interest, comprising loading a protein mixture which comprises the protein of interest to a filtration system comprising a feed tank, a reservoir tank, a filter, a three way valve comprising a feed tank valve connecting the filter to the feed tank and the reservoir tank valve connecting the filter to the reservoir tank, and a reservoir input connecting the feed tank and the reservoir tank.

14. The method of claim 12 or 13, wherein the reservoir tank valve is closed until the protein of interest is sufficiently concentrated.

15. The method of any one of claims 12 to 14, further comprising continually adding a protein mixture to the feed tank.

16. The method of any one of claims 12 to 15, wherein the protein mixture is directed from the feed tank to the reservoir tank.

17. The method of any one of claims 11 to 16, wherein the reservoir tank is connected to the filter.

18. The method of claim 17, wherein the filter comprises an in-line filtration membrane.

19. The method of claim 18, wherein the in-line filtration membrane is an ultrafiltration membrane.

20. The method of claim 18 or claim 19, wherein the in-line filtration membrane is polyvinylether, polyvinylalcohol, nylon, silicon, polysilicon, ultrananocrystalline diamond, diamond-like-carbon, silicon dioxide, titanium, silica, silicon nitride, polytetrafluorethylene, silicone, polymethacrylate, polymethyl methacrylate, polyacrylate, polystyrene, polyacrylamide, polymethacrylamide, polycarbonate, graphene, graphene oxide, polysaccharides, ceramic particles, poly(styrenedivinyl)benzene, polysulfone, polyethersulfone, modified polyethersulfone, polyarylsulfone, polyphenyl sulfphone, polyvinyl chloride, polypropylene, cellulose acetate, cellulose nitrate, polylactic acid, polyacrylonitrile, polyvinylidene fluoride, polypiperazine, polyamide-polyether block polymers, polyimide, polyetherimide, polyamide, regenerated cellulose, composite regenerated cellulose, or combinations thereof.

21. The method of any one of claims 18 to 20, wherein the filtration membrane has a molecular weight cutoff (MWCO) lower than from about 50 kD to about 5 kD, about 50 kD, about 40 kD, about 30 kD, about 20 kD, about 10 kD, or about 5 kD.

22. The method of claim 21, wherein the MWCO is lower than about 5 kD.

23. The method of any one of claims 1 to 22, wherein the mixture is allowed to flow until a desired filtered protein concentration is reached.

24. The method of claim 23, wherein the desired filtered protein concentration is from about 10 mg/mL to about 300 mg/mL, e.g., about 10 mg/mL, about 50 mg/mL, about 100 mg/mL, about 110 mg/mL, about 120 mg/mL, about 130 mg/mL, about 140 mg/mL, about 150 mg/mL, about 160 mg/mL, about 170 mg/mL, about 180 mg/mL, about 190 mg/mL, about 200 mg/mL, about 250 mg/mL, or about 300 mg/mL.

25. The method of claim 24, wherein the desired filtered protein concentration is about 150 mg/mL.

26. The method of any one of claims 23 to 25, wherein the protein viscosity is from about 0 cP to about 200 cP.

27. The method of claim 26, wherein the protein viscosity is from about 20 cP to about 60 cP.

28. The method of any one of claims 1 to 27, wherein the volume ratio between the volume of the feed tank and the volume of the reservoir is from about 1:2 to about 10:1, from about 1:2 to about 1:1, from about 1:1 to about 1:2, from about 1:1 about 1:3, from about 1:1 to about 1:4, from about 1:1 to about 1:5, from about 1:1 to about 1:6, from about 1:1 to about 1:7, from about 1:1 to about 1:8, from about 1:1 to about 1:9, or from about 1:1 to about 1:10.

29. The method of any one of claims 1 to 27, wherein the volume ratio between the volume of the feed tank and the volume of the reservoir is about 1:1, about 2:1, or about 5:1.

30. The method of any one of claims 16 to 29, wherein the protein mixture is directed to the reservoir tank and/or the filter using a diaphragm pump, rotary lobe pump, or a peristaltic pump.

31. The method of any one of claims 1 to 30, further comprising loading an initial protein mixture comprising a protein of interest that has not been filtered at least once to the feed tank prior to the continuous loading of the feed tank with the protein mixture comprising the protein of interest that has been filtered at least once (“retentate”).

32. The method of claim 31, wherein the initial protein mixture is added to the feed tank at a concentration of from about 1 mg/mL to about 30 mg/mL.

33. The method of claim 32, wherein the initial protein mixture is added to the feed tank at a concentration of about 5 mg/mL.

34. The method of any one of claims 1 to 33, wherein the process time is reduced by about 1%, about 5%, about 10%, about 20%, about 30%, about 40%, or about 50% as compared to a process time of a fed-batch concentration process.

35. The method of claim 34, wherein the process time is reduced by about 40% as compared to a process time of a fed-batch concentration process.

36. The method of any one of claims 1 to 33, wherein the process time is reduced by about 0.2 hours, about 0.4 hours, about 0.5 hours, about 0.6 hours, about 0.8 hours, or about 1.0 hours as compared to a process time of a fed-batch concentration process.

37. The method of claim 36, wherein the process time is reduced by about 0.5 hours as compared to a process time of a fed-batch concentration process.

38. The method of any one of claims 1 to 37, wherein the 1-2 μm particulate count is reduced by about 10%, about 20%, about 30%, about 40%, or about 50% as compared to a particulate count of a fed-batch concentration process.

39. The method of any one of claims 1 to 38, wherein the 5-10 μm particulate count is reduced by about 10%, about 20%, about 30%, about 40%, or about 50% as compared to a particulate count of a fed-batch concentration process.

40. The method of any one of claims 1 to 39, wherein the 10-25 μm particulate count is reduced by about 10%, about 20%, about 30%, about 40%, or about 50% as compared to a particulate count of a fed-batch concentration process.

41. The method of any one of claims 1 to 40, wherein the protein mixture comprises an antibody, antibody fragment, antigen-binding fragment, a fusion protein, a naturally occurring protein, a chimeric protein, or any combination thereof.

42. The method of claim 41, wherein the protein mixture comprises an antibody selected from IgM, IgA, IgE, IgD, and IgG.

43. The method of claim 42, wherein the protein mixture comprises an antibody and the antibody is an IgG antibody selected from IgG1, IgG2, IgG3, and IgG4.

44. The method of any one of claims 41 to 43, wherein the antibody comprises a dual variable domain immunoglobulin.

45. The method of any one of claims 41 to 43, wherein the antibody comprises a trivalent antibody.

46. The method of claim 41, wherein the antibody or antibody fragment comprises an anti-PD-1, anti-PD-L1 anti-CTLA4, anti-TIM3, anti-LAG3, anti-NKG2a, anti-ICOS, anti-CD137, anti-KIR, anti-TGFβ, anti-IL-10, anti-B7-H4, anti-GITR, anti-CXCR4, anti-CD73, anti-TIGIT, anti-OX40, anti-IL-8 antibody or antibody fragment thereof.

47. The method of any one of claims 41 to 46, wherein the protein mixture is derived from a bacterial, yeast, insect, or mammalian cell culture.

48. The method of claim 47, wherein the mammalian cell culture is a Chinese hamster ovary (CHO) cell culture.

49. The method of any one of claims 1 to 48, wherein the protein mixture is obtained from batch cell culture.

50. The method of any one of claims 1 to 49, wherein the protein mixture is obtained from fed batch cell culture.

51. The method of any one of claims 1 to 50, wherein the protein mixture is produced in a bioreactor.

52. The method of claim 51, wherein the protein mixture is produced in a single-use bioreactor.

53. The method of any one of claims 1 to 48, wherein the protein mixture is obtained from perfusion cell culture.

54. The method of claim 53, wherein the protein mixture is produced in a perfusion or TFF perfusion bioreactor.

55. The method of any one of claims 47 to 54, wherein the protein mixture is produced in a cell culture lasting from about 1 to about 60 days.

56. The method of claim 55, wherein the protein mixture is produced in a cell culture lasting about 25 days.

57. The method of any one of claims 1 to 54, wherein the protein mixture is added to the feed tank with a loading buffer.

58. The method of claim 57, wherein the loading buffer comprises amino acids, weak acids, weak bases, and/or sugars.

59. The method of any one of claims 1 to 58, further comprising formulating the protein into a pharmaceutical composition.

60. A protein prepared by the method of any one of claims 1 to 59.

61. A pharmaceutical composition comprising the protein of claims 1 to 60.

62. A method of administering the pharmaceutical composition of claim 61 to a subject in need thereof.

63. A method of treating a disease or condition in a subject in need thereof comprising administering to the subject the pharmaceutical composition of claim 61.

64. A system for concentrating a protein of interest, comprising:
a feed tank;
a reservoir tank connected to the feed tank by a first fluid pathway;
a filtration membrane connected to the reservoir tank by a second fluid pathway; and
a three-way valve, wherein the three-way valve is connected to the filtration membrane by a third fluid pathway, wherein the three-way valve is connected to the reservoir tank by a fourth fluid pathway, and wherein the three-way valve is connected to the feed tank by a fifth fluid pathway,
wherein the reservoir tank receives a protein mixture comprising the protein of interest from the feed tank via the first fluid pathway,
wherein the filtration membrane receives the protein mixture comprising the protein of interest from the reservoir tank via the second fluid pathway and filters the protein mixture, and
wherein the three-way valve receives retentate from the filter via the third fluid pathway and directs the retentate either to the reservoir tank via the fourth fluid pathway or to the feed tank via the fifth fluid pathway.

65. The system of claim 64, wherein the three-way valve directs the retentate to the reservoir tank if the total volume of the protein mixture within the system is less than the capacity of the reservoir tank, and wherein the three-way valve directs the retentate to the feed tank if the total volume of the protein mixture within the system is greater than the capacity of the reservoir tank.

66. The system of claim 64 or 65, further comprising a sensor configured to determine the total volume and/or concentration of the protein mixture within the system, wherein the three-way valve automatically directs the retentate either to the reservoir tank or to the feed tank based on feedback from the sensor.

67. The system of any one of claims 64 to 66, further comprising one or more diaphragm pumps, rotary lobe pumps, or peristaltic pumps.

68. The system of any one of claims 64 to 67, wherein wherein the filter comprises an in-line filtration membrane.

69. The system of claim 68, wherein the in-line filtration membrane is an ultrafiltration membrane.

70. The system of claim 69, wherein the in-line filtration membrane is polyvinylether, polyvinylalcohol, nylon, silicon, polysilicon, ultrananocrystalline diamond, diamond-like-carbon, silicon dioxide, titanium, silica, silicon nitride, polytetrafluorethylene, silicone, polymethacrylate, polymethyl methacrylate, polyacrylate, polystyrene, polyacrylamide, polymethacrylamide, polycarbonate, graphene, graphene oxide, polysaccharides, ceramic particles, poly(styrenedivinyl)benzene, polysulfone, polyethersulfone, modified polyethersulfone, polyarylsulfone, polyphenyl sulfphone, polyvinyl chloride, polypropylene, cellulose acetate, cellulose nitrate, polylactic acid, polyacrylonitrile, polyvinylidene fluoride, polypiperazine, polyamide-polyether block polymers, polyimide, polyetherimide, polyamide, regenerated cellulose, composite regenerated cellulose, or combinations thereof.
